(19) United States
(12) Patent Application Publication
(10) Pub. No.: US 2024/0207121 A1
(43) Pub. Date: Jun. 27, 2024

(54) TRANSFER TRAY AND PRIMING CART FOR NEONATAL CANNULATION AND RELATED METHODS

(71) Applicant: THE CHILDREN'S HOSPITAL OF PHILADELPHIA, Philadelphia, PA (US)

(72) Inventors: Joshua MARTIN, Providence, RI (US); Dana ZITNICK, Warwick, RI (US); Spencer BROWN, Cranston, RI (US); Dustin GAIDOS, Milton, MA (US); Joseph GORDON, Mansfield, MA (US); Alan W. FLAKE, Philadelphia, PA (US); Marcus Graeme DAVEY, Ardmore, PA (US)

(21) Appl. No.: 18/557,664

(22) PCT Filed: Apr. 28, 2022
(86) PCT No.: PCT/US2022/026848

§ 371 Date: Oct. 27, 2023

Related U.S. Application Data
(60) Provisional application No. 63/180,819, filed on Apr. 28, 2021.

(51) Int. Cl. A61G 11/00; A61M 1/36

(52) U.S. Cl. CPC A61G 11/001; A61M 1/3643; A61M 1/3653; A61M 2240/00

(57) ABSTRACT

Priming carts, transfer trays, priming circuits, blood circuits, and related methods are disclosed. A transfer tray for receiving a neonate comprises a body and a movable support assembly. The body has a first portion to receive a neonatal chamber assembly with a neonate therein and a second portion to receive a blood circuit. The blood circuit includes an oxygenator, a first conduit for transfer of blood from the neonate to the oxygenator, and a second conduit for transfer of blood from the oxygenator to the neonate. The movable support assembly is affixed to the body of the tray and is configured to receive the blood circuit. When the neonate is disposed on the first portion and is in liquid communication with the blood circuit, the transfer tray is movable from a first position on a first surface to a second position on a second surface.

### CROSS-REFERENCE TO RELATED APPLICATIONS

This application claims the benefit of U.S. Provision Application Ser. No. 63/180,819 filed Apr. 28, 2021, the contents of which is hereby incorporated by reference as if set forth in its entirety herein.

### TECHNICAL FIELD

The present disclosure relates generally to neonatal care, and more specifically to systems and methods related to improving viability of a premature neonate outside of the womb.

### BACKGROUND

Extreme prematurity is a leading cause of infant morbidity and mortality in the United States. Premature birth may occur due to any one of a multitude of medical reasons. Respiratory failure represents a common and challenging problem associated with extreme prematurity, as gas exchange in critically preterm neonates is impaired by structural and functional immaturity of the lungs. Even with medical advances in this field, there is still a high rate of chronic lung disease and other complications of organ immaturity in prematurely born children, particularly in neonates born prior to 28 weeks gestation.

### SUMMARY

The foregoing needs are met by the various aspects of priming carts, transfer trays, priming circuits, blood circuits, and related methods disclosed. According to an aspect of the disclosure, a transfer tray for receiving a neonate thereon may comprise a body and a movable support assembly affixed to the body. The body may have a first portion configured to receive a neonatal chamber assembly thereon, the neonatal chamber assembly configured to receive the neonate therein, and a second portion configured to receive a blood circuit thereon. The blood circuit may comprise an oxygenator, a first conduit for transfer of blood from the neonate to the oxygenator, and a second conduit for transfer of blood from the oxygenator to the neonate. The first conduit may have an arterial end portion configured to be placed in liquid communication with an artery of an umbilical cord of the neonate, and the second conduit having a venous end portion configured to be placed in liquid communication with a vein of the umbilical cord. The movable support assembly may be affixed to the body of the tray and be configured to receive the blood circuit thereon. When the neonate is disposed on the first portion and is in liquid communication with the blood circuit, the transfer tray may be movable from a first position, in which the transfer tray is disposed on a first surface, to a second position, in which the transfer tray is disposed on a second surface different from the first surface.

Optionally, the first surface may include a priming cart, and the second surface includes an incubator.

Optionally, the transfer tray may comprise a support member configured to receive the neonatal chamber assembly thereon, the support member causing the neonatal chamber assembly to be spaced from the transfer tray at a first height.

Optionally, the support member may be adjustable between having a first height and a second height different from the first height, the first and second heights being measured between the transfer tray and the neonatal chamber assembly.

Optionally, the transfer tray may comprise a plurality of support members.

Optionally, the body may define a receptacle defined thereon configured to receive and retain a first volume of liquid.

Optionally, the support assembly may comprise a support body attached there, the support body being configured to receive at least one sensor out of the group of oxygen sensor, flow meter, temperature sensor, pressure sensor, and bubble sensor.

Optionally, the support body may be movable relative to the support assembly.

Optionally, the support assembly may comprise an oxygenator retainer configured to releasably receive the oxygenator thereon.

Optionally, the oxygenator retainer may be movable relative to the support assembly.

Optionally, the transfer tray may comprise a first and a second pressure sensor disposed on the support assembly, the first pressure sensor being configured to measure pressure within the first conduit and the second pressure sensor being configured to measure pressure within the second conduit.

Optionally, the first and second pressure sensors may be movable along a vertical axis relative to the transfer tray toward and away from the transfer tray.

Optionally, the neonate may be configured to be disposed in a first plane spaced from the transfer tray along the vertical axis, and the first and second pressure sensors are configured to be moved into the first plane.

Optionally, the transfer tray may comprise a retention member on the body of the transfer tray, the retention member configured to perform at least one of: align the transfer tray with at least one of the first surface and the second surface; and preclude slidable movement of the transfer tray relative to at least one of the first surface and the second surface.

According to another aspect of the disclosure, a priming apparatus for priming a plurality of conduits of an external support system and for connecting a neonate to the external support system may comprise a surface configured to removably receive the neonate thereon; a blood circuit for connecting to the neonate; a priming circuit; a heater; and a pump. The blood circuit may comprise an oxygenator, a first conduit for transfer of blood from the neonate to the oxygenator, and a second conduit for transfer of blood from the oxygenator to the neonate, the first conduit having an arterial end portion and the second conduit having a venous end portion. The priming circuit may comprise a priming liquid source and a priming conduit in liquid communication with the blood circuit for transferring the priming liquid from the priming liquid source to the blood circuit. The heater may be configured to heat the priming liquid. The pump may be configured to pump the priming liquid from the priming liquid source through the priming conduit and through the blood circuit. The arterial end portion and the venous end portion of the blood circuit may be sized and configured to be disconnected from the priming conduit of the priming circuit and to be connected to an umbilical cord of the neonate, such that the arterial end portion is in liquid communication with an artery of the umbilical cord and the venous end portion is in liquid communication with a vein of the umbilical cord. When the blood circuit is connected to the umbilical cord, a liquid connection may be established through the neonate between the arterial end portion and the venous end portion, such that blood is permitted to be moved into the umbilical cord from the blood circuit, and blood from the neonate is permitted to be moved from the umbilical cord to the blood circuit.

Optionally, the priming apparatus may be a movable cart configured to be moved from a first location to a second location different from the first location.

Optionally, the priming apparatus may comprise at least one wheel along which the priming apparatus can be translated.

Optionally, the priming apparatus may comprise an upper portion that includes the surface configured to receive the neonate thereon, a lower portion spaced from the upper portion along a vertical axis, and a central portion disposed between the upper portion and the lower portion.

Optionally, the priming apparatus may be configured to have a first height and a second height greater than the first height, the first and second heights being measured along the vertical axis between the upper portion and the lower portion, the priming apparatus being movable between the first height and the second height.

Optionally, the priming apparatus may comprise at least one weight sensor on the surface, the at least one weight sensor being configured to detect a weight of the neonate received on the surface.

Optionally, the priming apparatus may comprise an oxygen sensor configured to measure a concentration of oxygen within at least one of the neonate, the priming circuit, and the blood circuit.

Optionally, the priming apparatus may comprise a temperature sensor configured to measure a temperature of a liquid within at least one of the priming circuit and the blood circuit.

Optionally, the priming apparatus may comprise a pressure sensor configured to measure a pressure of a liquid within at least one of the priming circuit, the blood circuit, and the neonate.

Optionally, the priming apparatus may comprise a first pressure sensor configured to measure a pressure within the first conduit of the blood circuit and a second pressure sensor configured to measure a pressure within the second conduit of the blood circuit, wherein the first and second pressure sensors are movable to be disposed in a plane in which the neonate is also disposed.

Optionally, the priming apparatus may comprise a gas tank configured to receive a predetermined gas mixture, wherein the oxygenator is configured to receive the predetermined gas mixture from the gas tank.

Optionally, the priming apparatus may comprise a flow controller is configured to receive the predetermined gas mixture from the gas tank and control the gas delivery to the oxygenator.

Optionally, the priming apparatus may comprise a power source configured to provide electrical power to the priming apparatus.

Optionally, the power source may include a battery.

Optionally, the priming liquid may include human blood.

Optionally, the priming liquid may include a crystalloid solution.

Optionally, the priming circuit may be configured to be connectable to a replacement oxygenator different from the oxygenator in the blood circuit and to move the priming liquid into the replacement oxygenator.

Optionally, the priming circuit may be configured to releasably connect with the blood circuit through a connection assembly, the connection assembly comprising: a blood circuit connector configured to receive the blood circuit therein; a priming circuit connector configured to receive the priming circuit therein; and a chamber defined between the blood circuit connector and the priming circuit connector configured to be placed into liquid communication with the blood circuit and the priming circuit when the blood circuit and the priming circuit are connected to the blood circuit connector and priming circuit connector, respectively.

According to another aspect of the disclosure, a connection assembly for releasably connecting a first conduit to a second conduit may comprise a body having a first end and a second end spaced from the first end along an axial direction; a first connector configured to receive the first conduit at the first end of the body; a second connector configured to receive the second conduit at the second end of the body; a carriage configured to be moved along the body between an unlocked position and a locked position, the carriage being movable along the axial direction in a first direction towards the first end and in a second direction opposite the first direction towards the second end; a resilient member configured to apply a force to the carriage to cause the carriage to move in one of the first and second directions; and a deformable release arm having a first position and a second position, where when in the first position, the deformable release arm precludes the carriage from being moved by the resilient member relative to the body, and when in the second position, the deformable release arm is positioned such that the carriage is permitted to be moved by the resilient member. When the carriage is moved from the unlocked position to the locked position, the carriage may be configured to carry one of the first conduit and the second conduit towards and into contact with the other of the first conduit and the second conduit, such that the first conduit and the second conduit are in liquid communication with each other and a liquid-tight seal is formed between the first conduit and the first connector and between the second conduit and the second connector.

Optionally, the resilient member may be a spring.

Optionally, when the carriage is in the unlocked position, the spring may be in tension and may apply a biasing force on the carriage towards the locked position.

Optionally, the deformable release arm may include a projection extending therefrom, the body defines a shoulder configured to be contacted by the projection, and the projection is configured to be moved out of contact with the shoulder when the deformable release arm is moved from the first position to the second position.

Optionally, the deformable release arm may be movable from the first position to the second position by contacting a release member on a manifold, the manifold being configured to slidably receive the connection assembly thereon.

Optionally, when the connection assembly is in the unlocked configuration, the first conduit may be received in the first connector but not form a liquid-tight seal with the first connector, and when the connection assembly is in the locked configuration, the first conduit may be received in the first connector and form a liquid-tight seal with the first connector.

Optionally, the first conduit may be included in a blood circuit configured to connect to a neonate, and the second conduit may be included in a priming circuit configured to receive a priming liquid therein, wherein when the connection assembly is in the locked configuration, the blood circuit may be in liquid communication with the priming circuit, and the priming liquid may be movable between the blood circuit and the priming circuit.

According to another aspect of the disclosure, a method of cannulating an umbilical cord of a neonate on a priming apparatus is disclosed. The priming apparatus may comprise a blood circuit comprising an arterial end, a venous end opposite the arterial end, a blood conduit extending between the arterial and venous ends, and an oxygenator disposed in line with the conduit between the arterial end and the venous end. The priming apparatus may comprise a priming circuit comprising a first end, a second end opposite the first end, and a priming conduit extending between the first and second ends, wherein the first end of the priming conduit is configured to releasably connect with the arterial end of the blood conduit, and the second end of the priming conduit is configured to releasably connect with the venous end of the blood conduit, such that the blood conduit is in liquid communication with the priming conduit, the blood circuit and the priming circuit being configured to receive a priming liquid. The method may include steps of: providing the neonate on an upper surface of the priming apparatus; calculating a weight of the neonate via a weight sensor on the upper surface of the priming apparatus; connecting the arterial end of the blood conduit to an artery in the umbilical cord of the neonate; and connecting the venous end of the blood conduit to a vein in the umbilical cord of the neonate. When at last one of the arterial and the venous ends of the blood conduit are connected to the umbilical cord, the blood circuit may not be in liquid communication with the priming circuit.

Optionally the method may include steps of: calculating a weight of the neonate by having a scale on the floor that an assistant is standing on. The scale is tared prior to the cannulation procedure but with the assistant standing on it. During the cannulation, the assistant lifts the patient out of the chamber (to fill the umbilical vein with blood to aid in cannulation) and the weight measurement is collected.

According to another aspect of the disclosure, a method of priming a blood circuit with a priming liquid is disclosed. The blood circuit may be configured to be connected to a neonate, the blood circuit may comprise a first end, a second end opposite the first end, a blood conduit extending between the first and second ends, and an oxygenator disposed in line with the blood conduit. The method may comprise the steps of: connecting the first end of the blood circuit to a first end of a priming conduit of a priming circuit, such that the blood conduit and the priming conduit are in liquid communication with each other; connecting the second end of the blood conduit to a second end of the priming conduit, the second end being spaced from the first end of the priming conduit, the priming conduit extending between its first and second ends; receiving the priming liquid into the priming conduit from a priming liquid source; actuating movement of the priming liquid within the priming conduit into the blood conduit by pumping the priming liquid toward the connected blood conduit; after receiving the priming liquid into the blood conduit, discharging the priming liquid from the blood conduit into the priming conduit, the priming liquid having passed through the blood conduit and the oxygenator prior to being discharged into the priming conduit; and heating the priming liquid by contacting a heater to the priming conduit.

Optionally, the priming liquid may be pumped through the priming conduit towards one of the first end and the second end into the respective connected first end and second end of the blood conduit, and the priming liquid may be discharged into the priming conduit from the blood conduit at the other of the first end and the second end of the priming conduit.

Optionally, the pumping of the priming liquid may be actuated by a peristaltic pump configured to operably contact the priming conduit.

Optionally, the pumping of the priming liquid may be actuated by a centrifugal pump configured to operably contact the priming conduit.

Optionally, the method may comprise moving the priming liquid from the priming liquid source into the priming conduit through a supply line connecting the priming liquid source to the priming conduit.

Optionally, the method may comprise introducing a gas into the oxygenator at a gas inlet, wherein at least a portion of the gas is introduced into the priming liquid as the priming liquid is moved through the oxygenator.

Optionally, the method may comprise measuring a temperature of the priming liquid in at last one of the priming conduit and the blood conduit.

Optionally, the method may comprise providing a signal to the heater to increase or decrease heat based on the measured temperature.

Optionally, the priming liquid may include at least one of blood plasma from the neonate, blood plasma from a human that is not the neonate, and synthetic plasma.

Optionally, the priming liquid may be a first priming liquid, and the method may comprise introducing a second priming liquid into the priming conduit from a second priming liquid source.

Optionally, the first priming liquid may include a crystalloid solution, and the second priming liquid may include human blood.

According to another aspect of the disclosure, a method of priming an oxygenator for use with a blood circuit connected to a neonate may include the steps of: connecting a liquid inlet of the oxygenator with a first end of a priming conduit; connecting a liquid outlet of the oxygenator with a second end of the priming conduit opposite the first end, such that a circuit is defined through the priming conduit and the oxygenator; and introducing a priming liquid through the priming conduit into one of the liquid inlet and liquid outlet of the oxygenator, such that the priming liquid is moved through the oxygenator and is discharged from the oxygenator back into the priming conduit from the other of the liquid inlet and liquid outlet.

Optionally, the method may comprise heating the priming liquid to a predetermined temperature and measuring the temperature of the priming liquid.

Optionally, the method may include introducing a gas from a controlled gas source into a gas inlet of the oxygenator, passing the gas through the oxygenator, and discharging the gas from the oxygenator at a gas outlet.

Optionally, the priming liquid may be a first priming liquid, and the method may comprise introducing a second priming liquid.

Optionally, the first priming liquid may be different from the second priming liquid.

According to another aspect of the disclosure, a method of replacing an oxygenator within a blood circuit is disclosed. The blood circuit may comprise an arterial end, a venous end opposite the arterial end, may further comprise the oxygenator disposed in line with a blood conduit between the arterial and the venous ends, a first portion of the blood conduit between the arterial end and the oxygenator, and a second portion of the blood conduit between the oxygenator and the venous end. The oxygenator may comprise a liquid inlet for receiving neonatal blood from the first portion of the blood conduit into the oxygenator, a liquid outlet for discharging the neonatal blood from the oxygenator into the second portion of the blood conduit, a gas inlet for receiving a gas into the oxygenator, and a gas outlet for discharging a gas from the oxygenator. The disclosed method may include the steps of: connecting a bypass to the blood circuit by placing a first end of the bypass into liquid communication with the first portion of the blood conduit, and placing a second end of the bypass into liquid communication with the second portion of the blood conduit, the bypass defining a lumen between its first and second ends that is configured to receive the neonatal blood from the blood conduit into the first end of the bypass and to discharge the neonatal blood into the blood conduit from the second end; moving the neonatal blood through the bypass such that the neonatal blood is moved from the first portion of the blood conduit into the first end of the bypass, through the bypass, and out of the second end of the bypass into the second portion of the blood conduit without moving through the oxygenator, wherein moving the neonatal blood through the bypass includes precluding movement of the neonatal blood into the oxygenator; disconnecting the oxygenator from the blood conduit such that the oxygenator is not in liquid communication with the blood conduit; connecting a replacement oxygenator to the blood conduit, such that the first portion of the blood conduit is in liquid communication with a liquid inlet of the replacement oxygenator, and the second portion of the blood conduit is in liquid communication with a liquid outlet of the replacement oxygenator; moving the neonatal blood through the blood conduit from the arterial end of the blood conduit, through the replacement oxygenator, and toward the venous end of the blood conduit; and disconnecting the bypass from the blood conduit such that the bypass is not in liquid communication with the blood conduit.

Optionally, the method may comprise disconnecting a gas conduit from the gas inlet of the oxygenator and connecting the gas conduit to a gas inlet of the replacement oxygenator, such that gas from a gas source is configured to be moved to the replacement oxygenator.

### BRIEF DESCRIPTION OF THE DRAWINGS

The present application is further understood when read in conjunction with the appended drawings. For the purpose of illustrating the subject matter, there are shown in the drawings exemplary aspects of the subject matter; however, the presently disclosed subject matter is not limited to the specific methods, devices, and systems disclosed. In the drawings:

FIG. 1 illustrates a perspective view of a priming cart with a transfer tray and neonatal chamber assembly thereon according to an aspect of this disclosure;

FIG. 2 illustrates a perspective view of the priming cart of FIG. 1 according to an aspect of this disclosure;

FIG. 3 illustrates another perspective view of the priming cart of FIG. 1;

FIG. 4 illustrates a perspective view of a priming circuit according to an aspect of this disclosure;

FIG. 5 illustrates a front perspective view of a priming cart with a priming circuit connected thereto according to an aspect of this disclosure;

FIG. 6 illustrates a perspective view of a blood circuit according to an aspect of this disclosure;

FIG. 7 illustrates a priming cart with a connected priming circuit thereon, with a transfer tray on the cart, and with a blood circuit connected to the priming circuit according to an aspect of this disclosure;

FIG. 8 illustrates a front perspective view of a connection interface between the blood circuit and the priming circuit according to an aspect of this disclosure;

FIG. 9 illustrates a flow chart of a method of connecting the blood circuit to the priming circuit according to an aspect of this disclosure;

FIG. 10 illustrates a perspective view of a connection assembly according to an aspect of this disclosure;

FIG. 11 illustrates another perspective view of the connection assembly of FIG. 10 with the cover removed;

FIG. 12 illustrates an exploded perspective view of a connection assembly according to another aspect of this disclosure;

FIG. 13 illustrates another exploded view of the connection assembly of FIG. 12 with the cover removed;

FIG. 14A illustrates a side cross-sectional view of a connection assembly in a partially connected configuration according to an aspect of this disclosure;

FIG. 14B illustrates a side cross-sectional view of the connection assembly of FIG. 14B shown in a fully connected configuration according to an aspect of this disclosure;

FIG. 14C illustrates a side cross-sectional view of a blood circuit connector of the connection assembly of FIG. 14A according to an aspect of this disclosure;

FIG. 15 illustrates a perspective view of a manifold for receiving a connection assembly according to an aspect of this disclosure;

FIG. 16 illustrates a perspective view of the manifold of FIG. 15 with a connection assembly attached thereto according to an aspect of this disclosure;

FIG. 17A illustrates a connection assembly in a first position relative to the manifold according to an aspect of this disclosure;

FIG. 17B illustrates the connection assembly and manifold of FIG. 17A, with the connection assembly being in a second position relative to the manifold;

FIG. 17C illustrates the connection assembly and manifold of FIG. 17A, with the connection assembly being in a third position relative to the manifold;

FIG. 18 illustrates a perspective view of a cradle for receiving the manifold and connection assemblies according to an aspect of this disclosure;

FIG. 19 illustrates a perspective view of a transfer tray with a neonatal chamber assembly having a neonate and with components of the blood circuit thereon according to an aspect of this disclosure;

FIG. 20 illustrates a perspective view of the transfer tray of FIG. 19;

FIG. 21 illustrates another perspective view of the transfer tray of FIG. 19;

FIG. 22 illustrates a portion of the transfer tray of FIG. 19 showing a support assembly according to an aspect of this disclosure;

FIG. 23 illustrates a perspective view of a portion of a transfer tray with a support assembly and with a blood circuit connected thereto according to an aspect of this disclosure;

FIG. 24 illustrates a perspective view of a portion of a transfer tray with a support assembly and with a blood circuit connected thereto according to another aspect of this disclosure;

FIG. 25 illustrates a perspective view of a blood circuit with a replacement oxygenator therein

FIG. 26 illustrates a flow chart depicting a method of priming a replacement oxygenator and replacing the oxygenator in the blood circuit according to an aspect of this disclosure;

FIG. 27 illustrates a top plan view of a drape of FIG. 7; and

FIG. 28 illustrates a therapy flow chart in accordance with one embodiment of the present invention.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

Disclosed are devices and methods related to cannulating a neonate after removing the neonate from the mother's womb. When a neonate needs to be removed from the womb prematurely, the neonate may need additional time to develop, preferably in an environment similar to that of a natural womb. For purposes of this disclosure, “neonate” can include a neonate that was prematurely removed from the womb, as well as a full-term baby that may need additional development time in a womb-like environment. The terms “neonate” and “baby” or “fetonate” may be used interchangeably throughout this disclosure.

The development of a system that could support normal neonatal growth and organ maturation for even a few weeks could significantly reduce the morbidity and mortality of extreme prematurity and improve quality of life in survivors. There are shortcomings with existing mechanisms for supporting premature neonates. Existing attempts to achieve adequate oxygenation of the neonate in animal models have been limited by circulatory overload and cardiac failure. The known systems suffer from unacceptable complications, such as circulatory failure and contamination. Accordingly, systems and methods for providing extracorporeal support for a premature neonate, or neonates (preterm or term) with inadequate respiratory gas exchange to support life, due to a spectrum of conditions/disorders, may improve viability.

After the neonate is removed from the mother's womb via cesarean or vaginal delivery, the neonate can be connected to an external support system that can provide necessary nutrients to the neonate, maintain a womb-like physical environment, and allow the neonate to develop therein. In such an external support system, the neonate may be connected to an extracorporeal blood, oxygenation, and/or filtration circuit, through which nutrients can be delivered to the neonate, waste can be removed, and desired levels of oxygen and temperature can be maintained. Some of the above systems can be connected to the neonate via one or more blood vessels in the neonate's umbilical cord. After removing the neonate from the womb, one or more blood vessels in the umbilical cord can be cannulated and placed in liquid communication with one or more external circulation systems listed above and that are described in further detail throughout this application.

When the neonate is first removed from the womb, it needs to be placed in a suitable environment so that it can be connected to the external systems. This environment can advantageously be in close proximity to the womb, so that the neonate can be placed therein immediately after removal from the womb. This decreases the time the neonate is exposed to non-womb-like conditions. The neonate's umbilical cord vessels can then be cannulated and connected to the one or more external systems. After cannulation, the neonate can be transferred to a main console in which the neonate will remain for the duration of its development. The main console may be, or may include, an incubator portion configured to provide preferred environmental parameters (e.g., temperature, light, etc.) for desired neonatal development. It may be advantageous to perform the cannulation and any related procedures as quickly as possible after removing the neonate from the womb. In certain embodiments, the clinician cannulates the umbilical cord vessels to the device about 10 minutes or less after removal from the womb, or about 5 minutes or less after removal from the womb, or about 3 minutes or less after removal from the womb, or about 2 minutes or less after removal from the womb. However, the main console may be located at an undesirable distance from the womb, for example outside of the operating room in which the removal of the neonate can occur. The main console may also be unwieldy and difficult to maneuver into close proximity to the womb. In some cases, the main console may include various hardwired connections to the medical facility, such as gas and electrical lines. Thus, it may not be practicable to have the main console in the sterile field. A longer time may be required to transfer the neonate directly from the womb to the main console. This may not be preferable because the longer the transfer process takes, the greater the delay of cannulating the umbilical cord. Long delays are not desired because risk of long-term injuries, developmental setbacks, or neonatal death increase with increased duration of the neonate being outside of the womb-like environment and not connected to an external oxygenating and/or feeding system.

As such, disclosed herein are systems and methods for receiving the neonate out of the womb that allow for timely cannulation of the umbilical cord vessels, placement of the neonate in a womb-like environment, and eventual transfer to the main console. Also disclosed are devices and methods for preparing the components necessary for the cannulation process and for proper operation of one or more external support system.

Certain terminology is used in the description for convenience only and is not limiting. The words “axial,” “vertical,” “transverse,” “left,” “right,” “above,” “below;” “longitudinal,” “transverse,” and “rotational” designate directions in the drawings to which reference is made. The term “substantially” is intended to mean considerable in extent or largely but not necessarily wholly that which is specified. The terminology includes the above-listed words, derivatives thereof and words of similar import.

The term “plurality,” as used herein, means more than one. The singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

### Priming Cart

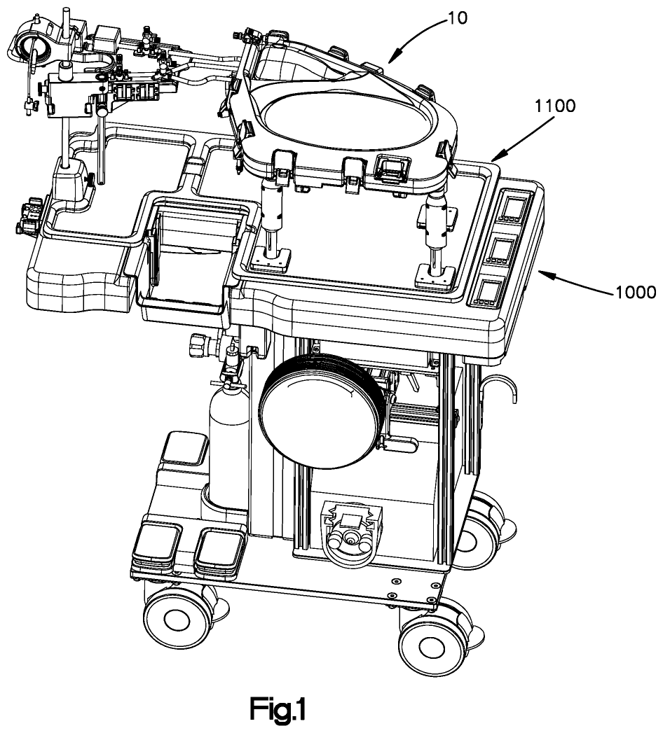

Referring, generally, to FIG. 1, a cart 1000 is depicted for receiving the neonate thereon and for preparing components of the external systems. The cart 1000 performs at least one of the following functions: provides a platform for priming the blood circuit; allows the clinicians to prepare the cannulas for use; support the neonatal chamber assembly during the cannulation procedure; and/or enables the transfer of the neonate patient in the neonatal chamber assembly to the console (not shown in FIG. 1). The cart 1000 can receive a transfer tray 1100 thereon, which can itself receive a neonatal chamber assembly 10. The neonatal chamber assembly 10 may be substantially similar to, or the same as, one or more neonatal chamber assemblies 10 described elsewhere in this application unless indicated otherwise. The neonate may be placed into the neonatal chamber assembly 10 upon removal from the womb. The neonate's umbilical cord can be measured via ultrasound prior to delivery. The measurement of the neonate's umbilical cord is used to determine the size of the cannula. The neonate's umbilical cord can then be cannulated to connect the neonate to a blood circuit 1200 (described below) to allow blood to flow from the neonate into and through the blood circuit 1200 and back into the neonate. After the neonatal umbilical cord has been connected to the blood circuit 1200, the neonate can be transferred to the main console (not shown). The neonate can remain in the neonatal chamber assembly 10 while transferred to the main console. In some aspects, the entire tray 1100 with the neonatal chamber assembly 10 can be moved from the cart 1000 to the main console. The cart 1000 can be selectively movable between different positions. For example, the cart 1000 can be moved into desired proximity with the womb to allow for sterile and quick transfer of the neonate from the womb to the cart 1000. The cart 1000 can then be moved to a desired proximity with the main console to facilitate transfer of the neonate into the main console.

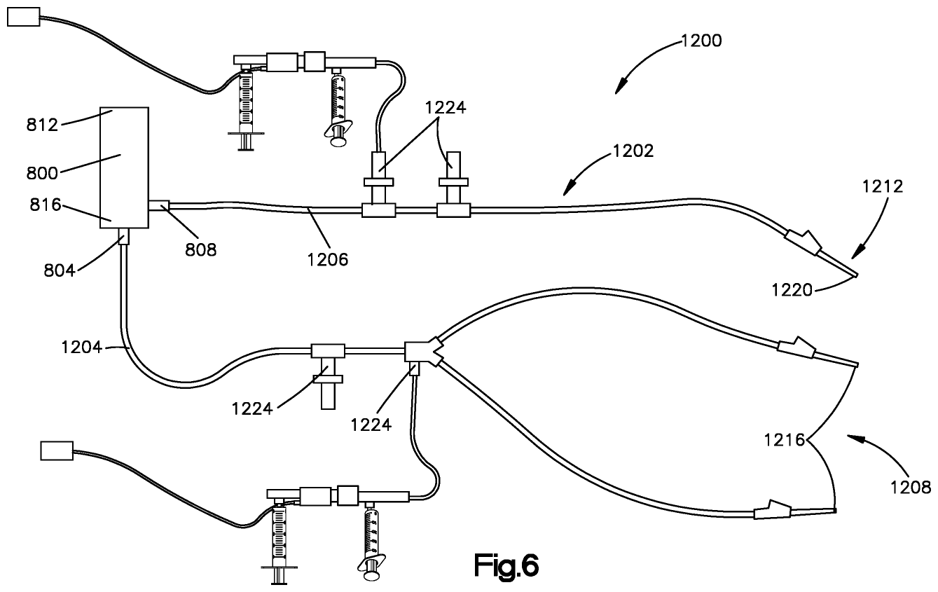

The cart 1000 may also include systems and components for preparing the blood circuit 1200 (an embodiment of which is shown in FIG. 6) for connection to the neonate. Such preparation may include priming components of the blood circuit 1200, such as tubing, oxygenators, and sensors, with suitable priming liquid which are described in detail below. Embodiments of the cart 1000 and the tray 1100 are described in detail below.

Referring to FIGS. 1-3, a cart 1000 includes an upper portion 1004, a lower portion 1030 spaced from the upper portion 1004, and a central portion 1018 spaced between the upper portion 1004 and the lower portion 1030. The upper portion 1004 may serve as a tabletop for the cart 1000 and is configured to receive the neonate thereon. The lower portion 1030 may serve as a structural base of the cart 1000 to maintain the cart 1000 in a sturdy and upright manner. For purposes of this disclosure, embodiments of the cart 1000 and the tray 1100 may be described with reference to a vertical axis 1001, a longitudinal axis 1002, and a transverse axis 1003, with each one of the vertical, longitudinal, and transverse axes 1001, 1002, and 1003 being orthogonal to both of the others of vertical, longitudinal, and transverse axes 1001, 1002, and 1003. The vertical axis 1001 may be defined as being parallel to the direction of gravity.

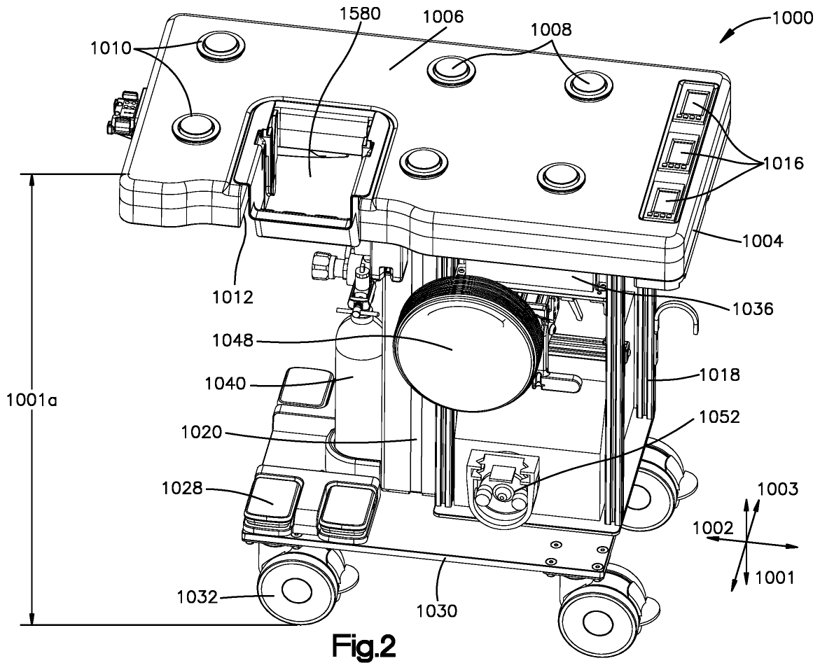

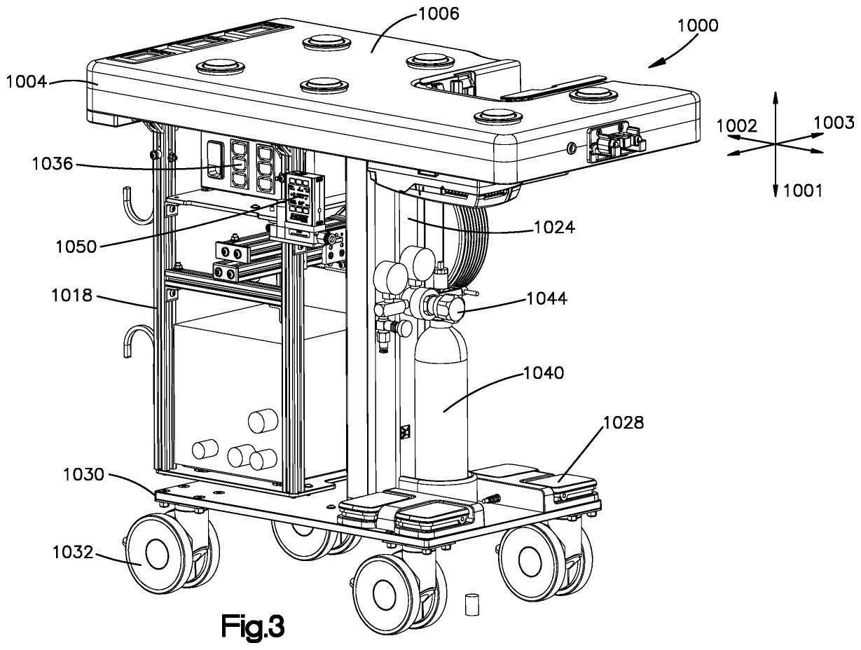

The upper portion 1004 may define an upper surface 1006 thereon (shown in FIGS. 2 and 3). In some aspects, the upper surface 1006 may be substantially planar in a plane defined by the longitudinal and lateral axes 1002 and 1003. It will be appreciated that in some aspects, the upper surface 1006 may instead be angled relative to the longitudinal and/or the transverse axes 1002 and 1003. The upper surface 1006 may receive the neonate thereon onto the cart 1000. In some aspects, the neonate may be placed into a neonatal chamber assembly 10 (see FIG. 1). In some aspects, the tray 1100 (shown in FIG. 1) may be removably disposed on the upper portion 1004 of the cart 1000. The tray 1100 may be in contact with the upper surface 1006 or, alternatively, the tray 1100 may be adjacent to the upper surface 1006 and spaced from the upper surface 1006 by one or more components along the vertical axis 1001.

The cart 1000 may include one or more sensors configured to measure and/or calculate a characteristic of the neonate. The cart 1000 may include a support 1008 mounted on a scale comprising weight sensors to measure the weight of the neonate. The supports 1008 may be resting on a scale structure withing upper portion 1004. It should be appreciated that the support(s) can be weight sensors themselves comprising the scale. It should be appreciated that the weight sensors comprising the scale can be calibrated to accurately detect and measure the neonatal weight. It should further be understood that the weight sensors comprising the scale may detect and measure a weight and then transmit the detected measurement(s) to a processor (not shown) that is configured to perform algorithmic processes to determine the weight of the neonate based on, at least in part, on the detected measurement by the scale. The scale may be disposed on or in the upper portion 1004 of the cart. The supports 1008 may be adjacent to, defined on, and/or extend from the upper surface 1006 along the vertical axis 1001 away from the cart 1000. In some aspects, the cart 1000 may include a plurality of supports 1008. As shown in the exemplary aspects of FIGS. 1-3, the cart 1000 may include six supports 1008, although it should be understood that another suitable number of supports can be utilized, such as 1, 2, . . . , 12, or another quantity of supports 1008. In aspects that the scale include a plurality of weight sensors, the processor may receive detected measurements from one or more of the plurality of weight sensors and calculate the weight of the neonate based on the detected measurements. The calculated weight of the neonate may be displayed on the cart 1000 (for example, on a display 1016 shown in FIG. 2 and explained in detail below) and/or transmitted electronically to a display component (not shown) separate from the cart 1000.

The upper portion 1004 is spaced from the lower portion 1030 along the vertical axis 1001. The central portion 1018 is disposed between the upper portion 1004 and the lower portion 1030. The central portion 1018 may serve as a structural frame, and it should be understood that the central portion 1018 should be sufficiently dimensioned and sturdy to support the upper portion 1004 and any components placed on the upper portion 1004 (e.g., the tray 1100, the neonatal chamber assembly 10, etc.). The central portion 1018 may be configured to receive thereon or therein one or more components related to the cart 1000, the tray 1100, or the blood circuit 1200, as will be described below.

The central portion 1018 may be adjustable in size based on desired use. Generally, the cart 1000 may define a height 1001 a measured along the vertical axis 1001 from the surface on which the cart 1000 is positioned (shown in FIG. 2). The height 1001a of the cart 1000 may be adjustable to correspond to steps of the neonatal removal, cannulation, and transfer processes and to complement respective heights of the individuals using the cart 1000 and to align the top surface of the cart 1006 with the works surface of the console (not shown). In some aspects, the upper portion 1004 may include a height adjustment assembly 1020 configured to change the height of the cart 1000. The height 1001a may be adjusted by moving the upper portion 1004 along the vertical axis 1001 relative to the lower portion 1030. The height 1001a increases when the upper portion 1004 is moved away from the lower portion 1030, and the height 1001a decreases when the upper portion 1004 is moved toward the lower portion 1030. When the height 1001a is increased, the central portion 1018 may also increase in size, measured along the vertical axis 1001, and when the height 1001a is decreased, the central portion 1018 may decrease in size.

The height adjustment assembly 1020 may be manually actuated to selectively increase or decrease the height 1001a through the actuating foot pedals 1028. A user of the cart 1000 can raise or lower the upper portion 1004 relative to the lower portion 1030. The height adjustment assembly 1020 may include a movement actuator, which causes movement of the upper portion 1004 relative to the lower portion 1030, and an input device 1028, which causes actuation of the movement actuator. In some aspects, the input device 1028 may include an electronic component, and the user can actuate the height adjustment assembly 1020 via an electronic command that is processed by a computing device and is transmitted to the height adjustment assembly 1020. Suitable input devices 1028 may include a push button, lever, switch, and/or the like, and the height adjustment assembly 1020 may include a plurality of input devices 1028 to allow for multiple users to operate the height adjustment assembly 1020 and/or for operation of the height adjustment assembly 1020 from different locations respective to the cart 1000.

The movement actuator may include any suitable assembly that can cause movement of connected components. Exemplary movement actuators may include motors, magnetic field tracks, piezoelectric components, pneumatic cylinders, hydraulic cylinders, and/or the like. In some aspects, for example those depicted in FIGS. 1-3, the height adjustment assembly 1020 may include an electronic linear actuator. The cart 1000 may include 1, 2, 3, 4, or more input devices 1028, which, in FIGS. 1-3, are depicted as pedals that can be operated by a user's foot. In use, the user may depress one or more of the pedals to cause the upper portion 1004 to move rise (i.e., move along the vertical axis 1001 away from the lower portion 1030) and thus increase the height 1001a, to lower (i.e., move along the vertical axis 1001 toward the lower portion 1030) and thus decrease the height 1001a, or selectively to rise and lower. In some aspects, one or more input devices 1028 of the plurality of input devices 1028 may be actuated to increase the height 1001a while one or more other input devices 1028 of the plurality of input devices 1028 may be actuated to decrease the height 1001a. The central portion 1018 may also increase or decrease as the height 1001a is increased or decreased, respectively.

The lower portion 1030 may serve as a structural base and support for the cart 1000. The lower portion 1030 can contact the surface on which the cart 1000 stands. The lower portion 1030 may be designed such that the cart 1000 is selectively movable relative to the surface on which it stands (i.e., along the longitudinal and lateral directions 1002 and 1003) and fixed along the longitudinal and lateral directions 1002 and 1003. In some aspects, the lower portion 1030 may include a plurality of moving components, such as wheels, or casters, 1032. The wheels 1032 can be defined on or connected to the lower portion 1030 and extend along the vertical axis 1001 toward the surface on which the cart 1000 stands. Although FIGS. 1-3 depict exemplary embodiments having four wheels 1032, it will be appreciated that the cart 1000 may include any suitable number of wheels 1032, such as 2, 3, 4, . . . 8, or another suitable quantity of wheels 1032. The wheels 1032 may be toggleable between locked and unlocked positions to selectively preclude and permit, respectively, rolling motion thereof. When the wheels 1032 are unlocked, the cart 1000 may be easily translated, via rolling motion of the wheels 1032, in the longitudinal and/or lateral directions 1002 and 1003 along the surface on which the cart 1000 stands. When the wheels 1032 are locked, their respective rolling motions are precluded, decreasing the translational mobility of the cart 1000 in the longitudinal and lateral directions 1002 and 1003. Locking the wheels 1032 may be advantageous when transferring and securing the neonate to the cart 1000, when preparing components of the blood circuit 1200, when transferring the transfer tray to the console, and/or when cannulating the neonate on the cart 1000. Keeping the cart 1000 stationary by precluding translational motion thereof decreases chances of human error due to unintended movement of the cart 1000 and/or any components affixed thereto or placed thereon. It may be further advantageous to unlock the wheels 1032 to allow for translation of the cart 1000 so that the cart 1000 may be readily moved from a first location to a second location. In some cases, the first location may be the operating room, in which the neonate was removed from the womb, placed onto the cart 1000, and cannulated and connected to the blood circuit 1200. The second location may be in proximity to the main console into which the neonate can then be transferred from the cart 1000. It will be appreciated that additional components to facilitate movement of the cart 1000 are envisioned, such as a handle (not shown) that a user can grip to better steer the cart 1000 while moving it between locations.

The cart 1000 may be designed and configured to receive and retain various components thereon associated with neonatal umbilical cannulation. Components, such as a power source 1036, gas tank 1040, flow controller 1050, heater 1048, and/or pump 1052 (shown in FIGS. 2 and 3 and described in detail below) can be retained on the upper portion 1004, the lower portion 1030, the central portion 1018, or on one or more of the above portions of the cart 1000. The power source 1036 is configured to provide electrical power to the one or more components of the cart 1000, such as the heater 1048, the pump 1052, the one or more displays 1016, the height adjustment assembly 1020, or other components on the cart 1000. In some aspects, the power source 1036 may include a battery. In this or other aspects, the battery charging status, battery life, or other attributes may be shown on one or more displays 1016. As shown in the exemplary embodiments of FIGS. 2 and 3, at least some of the above components may be disposed on the lower portion 1030 and the central portion 1018. It will be appreciated that the components should be properly stored to allow for their intended functionality, as well as adequately secured to prevent unwanted movement or damage to the components during use of the cart 1000.

### Priming Circuit

When the neonate is removed from the womb, it needs to be cannulated and connected to an external circulation system as described above to form a circulation circuit that can move blood from the neonate through one or more components designed to impart desired characteristics to the blood and then back into the neonate. Prior to connecting the neonate to such systems, the circuits and components need to be primed to have the desired properties for preferred physiological development of the neonate. The components (e.g., the blood circuit 1200 that will be described below in detail) should be primed to receive therein the desired priming liquid having the preferred composition, consistency, temperature, pressure, and other parameters. Described herein are systems and methods for priming the components that will be connected to the neonate.

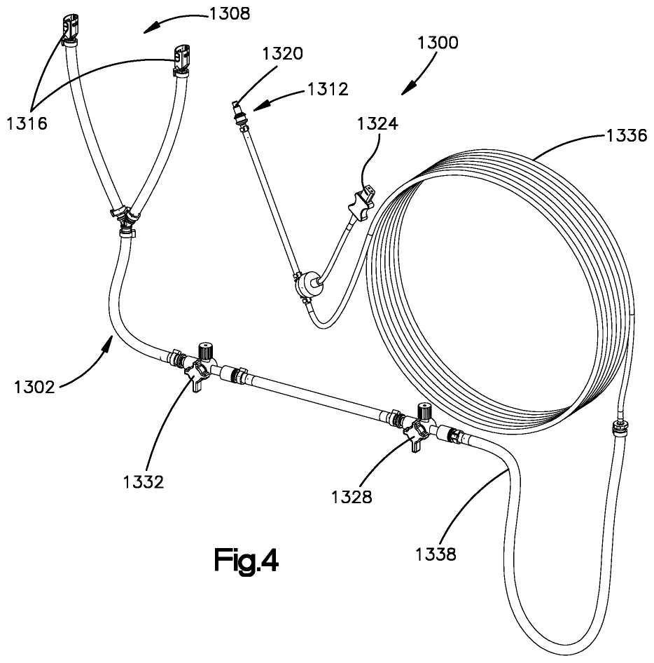

The cart 1000 may include a priming circuit 1300 configured to prime the blood circuit 1200 that can then be connected to the neonate. Referring to FIG. 4, a priming circuit 1300 includes a priming conduit 1302 that is configured to receive a priming liquid therein and to transport the priming liquid therethrough and to a connected circuit (e.g., a blood circuit 1200). The priming conduit 1302 may be a tube and may have a circular, oblong, or a differently shaped cross-section. The priming conduit 1302 is configured to receive a liquid therein and should be configured to retain and transfer the liquid therethrough. The priming conduit 1302 extends between a first end 1308 and a second end 1312 and defines a conduit length between the first and second ends 1308 and 1312. The conduit length includes the entire priming conduit 1302, including portions thereof. The priming conduit 1302 may include silicone, thermoplastic elastomer, fluoropolymer, and/or another suitable polymer or material. In some exemplary embodiments, the priming conduit 1302 may include polymeric tubing under the trade name Tygon. It should be understood that the selected material should be biocompatible and should be acceptable for use with blood components. The selected material should be suitable to receive and transfer heat to the liquid flowing therethrough. The selected material should also have desired gas exchange (or gas exchange preclusion) properties to facilitate gas transfer to and from the liquid in the priming conduit 1302 and/or preclude gas transfer so as to maintain a desired gas concentration in the liquid within the priming conduit 1302. The selected material should also be amenable to transferring heat therethrough to allow for heating of the priming liquid. In some aspects, the priming conduit 1302 may include one or more coatings thereon to facilitate liquid flow therethrough and/or neonatal development, such as anticoagulant or non-thrombogenic coatings, antibacterial coating, hydrophobic coating, and/or the like. The thickness of the wall defining the priming conduit 1302 should be sufficient to allow for effective heat transfer therethrough.

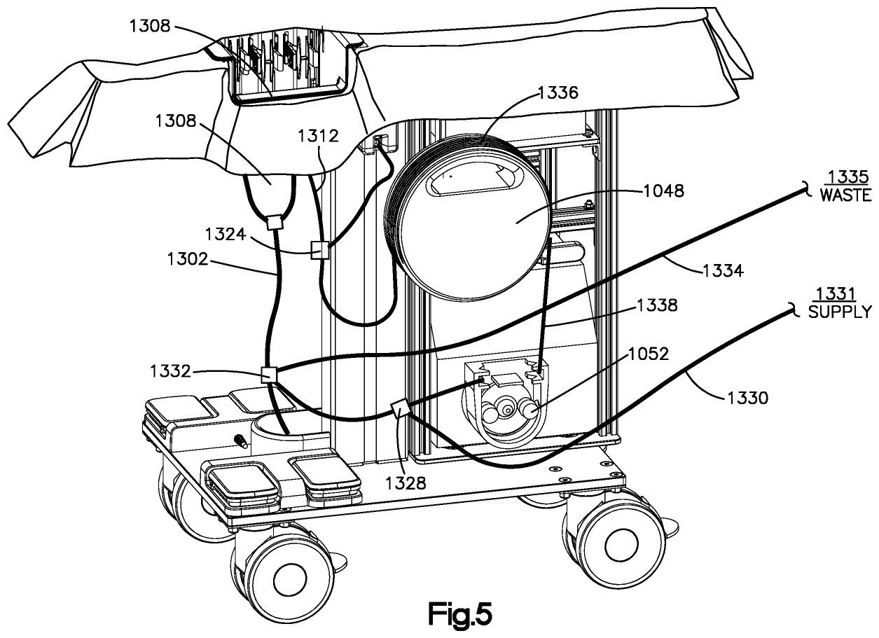

The priming conduit 1302 may include a portion thereof configured to receive heat and to transfer the received heat to the priming liquid in the priming conduit 1302. A heating portion 1336 (shown in FIGS. 4 and 5) may be configured to be placed adjacent to a heater 1048 (see FIG. 5) such that heat from the heater 1048 can be transferred to the heating portion 1336. As shown in the exemplary embodiment of FIG. 5, the heating portion 1336 may be wrapped around the heater 1048. The heating portion 1336 may wrap around the heater 1048 for multiple revolutions. It will be appreciated that the more heating portion 1336 contacts the heater 1048, the greater the surface area will be between the heating portion 1336 and the heater 1048, and thus the greater the amount of received heat will be from the heater 1048 to the heating portion 1336. Although the heating portion 1336 is depicted as a coil wrapped around the heater 1048, other embodiments are envisioned where the heating portion 1336 is arranged in a different way relative to a heater 1048. The heating portion 1336 may be an integrated and uniform part of the priming conduit 1302. In some aspects, the heating portion 1336 may be separable from the rest of the priming conduit 1302 and be connectable to the priming conduit 1302.

The priming conduit 1302 may include a pumping portion 1338 configured to receive priming liquid therein and to be contacted by a pump 1052 (see FIG. 5) to cause movement of the priming liquid through the priming conduit 1302. The pumping portion 1338 may be an integrated and uniform part of the priming conduit 1302 or, alternatively, in some aspects, the pumping portion 1338 may be separable from the rest of the priming conduit 1302 and be connectable to the priming conduit 1302. In some aspects, the priming conduit 1302 includes a pumping portion 1338 that is connectable, and placeable in liquid communication with, the heating portion 1336.

In some aspects, the priming conduit 1302 may comprise the same materials, dimensions, and consistency throughout. For example, the pumping portion 1338 may comprise similar or same characteristics and dimensions as the heating portion 1336. Alternatively, in some aspects, the priming conduit 1302 may vary in materials, dimensions, and/or consistency along portions thereof. For example, the heating portion 1336 may comprise a different material, or a different composition of materials, than the pumping portion 1338 and/or another portion of the priming conduit 1302. The heating portion 1336 may comprise one or more materials better suited for heat transfer than materials used elsewhere in the priming conduit 1302.

The priming conduit 1302 may have a constant wall thickness throughout the entire priming conduit 1302 between the first end 1308 and the second end 1312. Alternatively, the priming conduit 1302 may have a variable wall thickness along its length. In some aspects, the heating portion 1336 may have a different wall thickness from another portion of the priming conduit 1302, such as the pumping portion 1338. In some exemplary aspects, the priming conduit 1302 may have an inner diameter of approximately 3/16 inch, and the outer diameter may be approximately 5/16 inch. Subsequently, the wall thickness of such an exemplary priming conduit 1302 can be approximately 1/16 inch. In some aspects, the wall thickness of the heating portion 1336 may be smaller than a wall thickness of one or more portion of the rest of the priming conduit 1302. A smaller wall thickness may increase the efficiency and/or speed of heat transfer from the heater 1048 through the heating portion 1336 to the priming liquid flowing therethrough. However, in some circumstances, the thinner wall thickness that may be present at the heating portion 1336 may not be preferable elsewhere in the priming conduit 1302. For example, such thin walls could lead to undesired stresses on the material of the priming conduit 1302, such as those exerted on the priming conduit 1302 by the pump 1052 at the pumping portion 1338 during pumping of the priming liquid through the priming conduit 1302. As such, it may be preferable in some aspects to have different wall thicknesses at different portions of the priming conduit 1302. For example, in some aspects, the wall thickness of the priming conduit 1302 at the heating portion 1336 may be smaller than the wall thickness of the priming conduit 1302 at the pumping portion 1338. The relatively greater wall thickness at the pumping portion 1338 can better withstand the stresses exerted on the priming conduit 1302 by the action of the pump 1052 during operation.

As noted above, the priming conduit 1302 defines a length between the first end 1308 and the second end 1312. The length of the priming conduit 1302 may include a total of lengths of each portion of the priming conduit 1302. For example, the total length of the priming conduit 1302 may include the aggregate lengths of the heating portion 1336 and the pumping portion 1338. In some aspects, the heating portion 1336 may comprise a greater percentage of the conduit length than the pumping portion 1338. In some particular aspects, the heating portion 1336 may be longer than the pumping portion 1338 by a multiple of between 1 and 2, between 2 and 3, between 3 and 4, between 4 and 5, between 5 and 6, or another preferable multiple. In some aspects, the lengths of the heating portion 1336 and the portion 1338 may be approximately equal. In some aspects, the length of the pumping portion 1338 may be greater than the length of the heating portion 1336, for example by a multiple of between 1 and 2, between 2 and 3, between 3 and 4, between 4 and 5, between 5 and 6, or another preferable multiple. In some exemplary aspects, the priming conduit 1302 may be up to 2000 mm in length between the first end 1308 and the second end 1312. It should be appreciated that the specific length of the priming conduit 1302 may depend on the intended use, the components being utilized (e.g., the heater 1048), and on desired arrangement of the priming conduit 1302.

The priming circuit 1300 is configured to receive a priming liquid from a priming liquid supply source 1331 and to transfer the priming liquid through the priming circuit 1300 and to any of the connected components (for example, the blood circuit 1200 and the oxygenator 800, as will be described in detail below). The priming liquid may include blood components from the neonate, from the neonate's mother, or from a donor, and may include blood plasma. In some aspects, the priming liquid may include another suitable liquid that can be used to supply the neonate with sufficient water, electrolytes, and calories. It should be appreciated that the selected priming liquid should be biocompatible with the neonate and should preferably exhibit similar characteristics to physiological blood plasma compositions having, for example, similar concentrations of electrolytes, osmolality, and pH. In some aspects, the priming liquid may include a crystalloid solution. An exemplary suitable priming liquid may include an intravenous product under the trade name of PlasmaLyte (or Plasma-Lyte), which exhibits desirable characteristics as described above. It will be understood that the priming liquid may include a combination of different suitable liquids, and this disclosure is not limited to use with a particular priming liquid. For example, in some aspects, a priming liquid that is used may include a combination of a crystalloid (e.g., PlasmaLyte) with albumin. In some aspects, the priming liquid may include blood, for example, blood from an adult human. In some aspects, two priming liquids may be used, where a first priming liquid includes a crystalloid, and a second priming liquid includes human blood. The human blood may be treated prior to be used as priming liquid, for example, by washing, irradiating, and/or removing platelets therefrom. In one particular embodiment, the first priming liquid can be PlasmaLyte. In the same or an alternative embodiment, the second priming liquid can be human Type O negative blood.

The priming liquid is introduced into the priming circuit 1300 from a supply source 1331 that can be connected to the priming circuit 1300. With continued reference to FIG. 5, the supply source 1331 may include a bag configured to receive and retain a desired volume of priming liquid (not shown). A supply line 1330 can extend between the supply source 1331 and the priming circuit 1300. The supply line 1330 may be a tube, hose, or another suitable conduit for transporting the priming liquid therethrough. The supply line 1330 can be in liquid communication with the supply source 1331, such that the priming liquid can move, or be moved, from the supply source 1331 into the supply line 1330. At its other end, the supply line 1330 can be placeable in liquid communication with the priming conduit 1302, such that the priming liquid in the supply line 1330 is permitted to enter the priming conduit 1302. In some aspects, the priming conduit 1302 may include a supply connector 1328 configured to interface between the priming conduit 1302 and the supply line 1330 and to receive the supply line 1330 into liquid communication with the interior of the priming conduit 1302. The supply connector 1328 may include a movable valve, a Luer connector, a stopcock, a resealable membrane, a capped opening, or another suitable connector commonly utilized in the medical field. The supply connector 1328 may be selectively opened and closed to allow priming liquid to enter the priming conduit 1302 and preclude the priming liquid from entering the priming conduit 1302, respectively. The priming liquid can thus be introduced into the priming conduit 1302 of the priming circuit 1300 via the supply line 1330 from the supply source 1331. When a desired volume of the priming liquid has been introduced into the priming conduit 1302 (e.g., when the entire priming conduit 1302 is filled with the priming liquid between the first end 1308 and the second end 1312), the supply connector 1328 may be actuated to block any further priming liquid from entering the priming conduit 1302 from the supply line 1330 (i.e., take the supply source 1331 and the supply line 1330 out of liquid communication with the priming conduit 1302). The priming liquid that is in the priming conduit 1302 can be moved through the priming circuit 1300.

After the priming liquid has been received in and moved through the priming circuit 1300 for the desired sufficient duration, at least a portion of the priming liquid can be removed, or drained, from the priming circuit 1300. A waste line 1334 can be connected to the priming conduit 1302. A waste connector 1332 may be defined on the priming conduit 1302 and can interface with the priming conduit 1302 and the waste line 1334 and is configured to receive the waste line 1334 into liquid communication with the interior of the priming conduit 1302. The waste connector 1332 can be selectively actuated to open and close a flow path between the priming conduit 1302 and the waste line 1334, such that when the waste connector 1332 is open, the waste line 1334 is in liquid communication with the priming conduit 1302, and when the waste connector 1332 is closed, the waste line 1334 is not in liquid communication with the priming conduit 1302. The waste connector 1332 may include a movable valve, a Luer connector, a stopcock, a resealable membrane, a capped opening, or another suitable connector commonly utilized in the medical field. The waste line 1334 may extend from the waste connector 1332 to a waste receptacle 1335. The waste receptacle 1335 may be a bottle, bag, drain, or another suitable container or location to receive liquid medical waste.

When the waste connector 1332 and the supply connector 1328 are both in their respective closed positioned, flow of priming liquid between the priming conduit 1302 and each of the waste line 1334 and the supply line 1330, respectively, is precluded. The priming liquid can now be moved through the priming circuit 1300, specifically along the priming conduit 1302 between the first end 1308 and the second end 1312. The priming liquid in the priming conduit 1302 can be subjected to one or more physical or chemical changes. In some aspects, the priming liquid can receive heat from a heat source (e.g., the heater 1048). As shown in FIG. 5 and described above, a heating portion 1304 of the priming conduit 1302 is configured to receive heat from the heater 1048. The heater 1048 may be connected to the cart 1000. As shown in FIG. 5, the heater 1048 may be disposed on the central portion 1018. However, it will be appreciated that the heater 1048 may be alternatively disposed on the upper portion 1004, on the lower portion 1030, or partially disposed on two adjacent portions of the cart 1000. In some aspects, a plurality of heaters 1048 may be utilized, with each of the plurality of heaters 1048 being disposed to contact at a portion of the priming circuit 1300. The priming liquid that is positioned in the heating portion 1304 of the priming conduit 1302 can receive heat by conduction through the walls of the priming conduit 1302 that contact the heater 1048 at the heating portion 1304. It will be appreciated that other mechanisms of heating the priming liquid may be utilized, such as convection from a spaced heating source.

The priming liquid is movable through the priming circuit via one or more pumping mechanisms. With continued reference to FIG. 5, a pump 1052 may be disposed in connection with, or adjacent to, the priming conduit 1302 so as to cause movement of the priming liquid through the priming conduit 1302. The pump 1052 may be affixed to the cart 1000. As shown in FIG. 5, the pump 1052 may be disposed on the central portion 1018. However, it will be appreciated that the pump 1052 may be alternatively disposed on the upper portion 1004, the lower portion 1030, or spanning two adjacent portions. In some aspects, the cart 1000 may include a plurality of pumps 1052, with each of the plurality of pumps 1052 being configured to cause movement of the priming liquid through the priming circuit 1300 and/or another conduit connected to the cart 1000 (e.g., within the blood circuit 1200). The pump 1052 may be a positive displacement pump. Examples of suitable pumps may include peristaltic pumps, centrifugal pumps, gear pumps, progressive cavity pumps, rotary pumps, diaphragm pumps, impeller pumps, or other suitable pumps. In some aspects depicted in the exemplary figures (e.g., FIG. 5), the pump 1052 may be a peristaltic pump configured to contact the priming conduit 1302 to cause movement of the priming liquid therein towards either the first end 1308 or the second end 1312. As described above, the priming conduit 1302 may include a pumping portion 1338 configured to be contacted by the pump 1052. The pumping portion 1338 may exhibit favorable physical and structural parameters to withstand the stresses associated with peristaltic pumping.

The priming circuit 1300 is configured to introduce the priming liquid therein into one or more components to prime those components prior to use. In some aspects, before the blood circuit 1200 can be connected to the neonate via umbilical cannulation, the blood circuit 1200 may need to be primed with the priming liquid that has been adapted to have, or maintained to retain, desired physical and chemical characteristics. In some aspects, it may be desirable to maintain the temperature of the priming liquid being circulated in the priming circuit 1300. As described above, the heater 1048 can apply heat to at least a portion of the priming conduit 1302, and thus to the priming liquid therein. The priming circuit 1300 may include a temperature sensor 1324 (see FIGS. 4 and 5) configured to detect and measure the temperature of the priming liquid in the priming conduit 1302. The temperature sensor 1324 may include, or be connectable to, a computing device with a processor (not shown) to receive and record the temperature of the priming liquid. In some aspects, heater 1048 may be controlled to apply more or less temperature to the priming conduit 1302 to raise or lower, respectively, the temperature of the priming liquid. The heater 1048 may be controlled based on the measured temperature of the priming liquid by the temperature sensor 1324. If the temperature sensor 1324 detects that the temperature of the priming liquid is below a predetermined threshold, the temperature sensor 1324 (or a connected computing device) can cause the heater 1048 to apply more heat to the priming conduit 1302, and if the temperature sensor 1324 detects that the temperature of the priming liquid is above a different predetermined threshold, the temperature sensor 1324 (or the connected computing device) can cause the heater 1048 to apply less heat to the priming conduit 1302.

The priming circuit 1300 may be disposed on, or attached to, the cart 1000. The priming circuit 1300 may be designed such that the priming circuit 1300 is integrated with the cart 1000 in such a way that when the cart 1000 is moved between locations, the priming circuit 1300 is also moved with the cart 1000. In some aspects, the supply source 1331, waste destination 1335, or both can be affixed to the cart 1000 such that the supply source 1331 and/or the waste destination 1335 are also movable along with the cart 1000. Alternatively, the priming circuit 1300 may be releasably connectable to the supply source 1331 and/or the waste destination 1335, one or both of which being separable from and independent of the cart 1000.

The priming circuit 1300 may include one or more connectors thereon to facilitate releasably connection between the priming circuit 1300 and the one or more connectable components (e.g., the blood circuit 1200). Referring again to FIG. 4, the priming circuit 1300 may include a first end connector 1316 disposed at, on, or adjacent to the first end 1308 of the priming circuit 1300. The first end connector 1316 is configured to releasably place a connectable component into liquid communication with the interior of the priming conduit 1302 cannula. In some aspects, the first end connector 1316 may be releasably connectable to a corresponding component of a blood circuit 1200, as will be described below. In some aspects, the priming circuit 1300 may include a plurality of first end connectors 1316, for example, 2, 3, 4, or another suitable number of first end connectors 1316. The one or more first end connectors 1316 may include a movable valve, a Luer connector, a stopcock, a resealable membrane, a capped opening, or another suitable connector commonly utilized in the medical field. In some aspects, the one or more first end connectors 1316 may be connectable with corresponding connectors of another component (e.g., the blood circuit 1200) via a connection apparatus, assembly, or adapter, such as a connection assembly 1500 that will be described in detail below.

The priming circuit 1300 may include a second end connector 1320 disposed at, on, or adjacent to the second end 1312 of the priming circuit 1300. The second end connector 1320 is configured to releasably place a connectable component into liquid communication with the interior of the priming conduit 1302 cannula. In some aspects, the second end connector 1320 may be releasably connectable to a corresponding component of the blood circuit 1200, as will be described below. The second end connector may include a movable valve, a Luer connector, a stopcock, a resealable membrane, a capped opening, or another suitable connector commonly utilized in the medical field. In some aspects, the second end connector 1320 may be connectable with a corresponding connector of another component (e.g., the blood circuit 1200) via a connection apparatus, assembly, or adapter, such as a connection assembly 1500 as will be described below. The second end connector 1320 may be dimensioned or arranged similar to or the same as the one or more first end connectors 1316. In some aspects, the second end connector 1320 may be arranged and/or dimensioned such that the second end connector 1320 is different from the one or more first end connectors 1316. Such difference may be due to different complementing connectors to which each of the first end and second end connectors 1316 and 1320 are designed to connect. In some aspects, the difference in dimension and/or arrangement or orientation may preclude accidental mix up of connectors when making the described connections so as to reduce user error and to ensure that the first end connectors 1316 are connected to the desired respective complementary connectors while the second end connectors 1320 are connected to their respective complementary connectors. The difference in dimensions, arrangement, and/or orientation between the first end and second end connectors 1316 and 1320 may be such that a connection between incorrect components is not permitted due to the structure or arrangement of the respective components that are to be connected. In some aspects, the priming circuit 1300 may include a plurality of second end connectors 1320, for example, 2, 3, 4, or another suitable number of second end connectors 1320.

### Blood Circuit

The priming circuit 1300 is designed to be releasably connectable with a blood circuit 1200 to prime the blood circuit 1200 with the priming liquid having desired characteristics prior to connecting the blood circuit 1200 to the neonate via umbilical cannulation. Turning to FIG. 6, an exemplary blood circuit 1200 is depicted having a first end 1208 and a second end 1212 opposite the first end 1208. A blood conduit 1202 extends between the first end 1208 and the second end 1212. The blood conduit 1202 is configured to receive a liquid therein, such as priming liquid from the priming circuit 1300 and neonatal blood from the neonate.

The blood conduit 1202 may include silicone, thermoplastic elastomer, fluoropolymer, and/or another suitable polymer or material. In some exemplary embodiments, the blood conduit 1202 may include polymeric tubing under the trade name Tygon. It should be understood that the selected material should be biocompatible and should be acceptable for use with blood and plasma components. The selected material should have desired gas exchange properties to facilitate gas transfer to and from the liquid in the blood conduit 1202 and/or preclude gas transfer so as to maintain a desired gas concentration in the liquid within the blood conduit 1202. In some aspects, the blood conduit 1202 may comprise the same or similar materials, and exhibit the same or similar characteristics, as the priming conduit 1302. In some aspects, the blood conduit 1202 may include one or more coatings thereon to facilitate liquid flow therethrough and/or neonatal development, such as anticoagulant coating, non-thrombogenic coating, antithrombogenic coating, antibacterial coating, hydrophobic coating, and/or the like.

The blood circuit 1200 can include a gas exchange mechanism, such as an oxygenator 800. The oxygenator 800 is configured to introduce oxygen to a flow of liquid therethrough, for example, to the priming liquid or to the neonatal blood. The oxygenator 800 can be designed to remove one or more gases from the liquid flowing therethrough, while imparting one or more gases to the liquid. It will be understood that the oxygenator 800 and/or other components of the blood circuit 1200 may be adjusted to result in a desired range of the different gases that can be present in, or introduced into, the priming liquid or the neonatal blood. It will be further appreciated that the blood circuit 1200 may include a plurality of oxygenators 800 arranged serially and/or in parallel relative to one another. Various suitable oxygenator designs can be utilized, and this disclosure is not limited to a particular type of oxygenator. As shown in FIG. 6, the oxygenator 800 may include a liquid inlet 804 at which the liquid flowing through the blood conduit 1202 (e.g., priming liquid or neonatal blood) can enter the oxygenator 800. The liquid that enters the oxygenator 800 may flow through one or more flow paths within the oxygenator (not shown) and exit the oxygenator at a liquid outlet 808, through which the liquid moves from the oxygenator into the blood conduit 1202. In some aspects, the liquid inlet and outlet 804 and 808 may be reversed in function based on the flow of liquid; for example, if liquid is flown from the first end 1208 of the blood conduit 1202 towards the second end 1212 of the blood conduit 1202, then the liquid enters the oxygenator 800 at the liquid inlet 804 and exits the oxygenator at the liquid outlet 808; however, if liquid is flown in a reverse direction from the second end 1212 towards the first end 1208, then the liquid enters the oxygenator 800 at the liquid outlet 808 and exits the oxygenator at the liquid inlet 804. As such, it should be appreciated that the nomenclature does not limit the functionality of the oxygenator 800 or the blood circuit 1200, and the liquid inlet and outlet 804 and 808 are defined as they are for ease of reference only. In some aspects, ports can be incorporated into the oxygenator on the blood inlet or blood outlet sides to allow pressure sensors to be connected to sense the blood pressure on one or both sides of the gas exchange medium.

The oxygenator 800 can receive one or more gases therein at a gas inlet 812. A sweep gas can be passed through the gas inlet 812. The sweep gas can be introduced in a desired direction 801 from the inlet 804 to the outlet 808. The sweep gas can remove any ambient air from the oxygenator 800. The sweep gas can include about 0% to about 100% O₂. The sweep gas can include about 0% to about 50% CO₂. The sweep gas can include about 0% to about 100% N₂. In one particular embodiment, the sweep comprises oxygen, carbon dioxide, and nitrogen. In this or another embodiment, the sweep gas can pass through the oxygenator 800 at a flow rate of about 30 mL/min to about 40 mL/min. The sweep gas flow rates can be adjusted on the system monitor (not shown). The sweep gas can regulate the gas levels in the blood delivered to the neonate. In one aspect, the oxygen is absorbed into the neonate blood and the carbon dioxide is removed. The composition of the sweep gas can be adjusted to achieve desired levels of blood oxygen and carbon dioxide saturation in the neonate blood or in the neonate exhalation. In some embodiments, it is desirable to have about 2% to about 5% CO₂ in the exhaled gas.

Gas that enters the oxygenator 800 can be introduced into the liquid flowing through the oxygenator 800. Gas that is removed from the liquid as the liquid passes through the oxygenator 800 can be removed from the oxygenator 800 via a gas outlet 816. The gas inlet 812 may be connected to a gas source via a suitable gas conduit and flow control element. In some aspects, the gas source may include a portable tank 1040 (see FIGS. 2 and 3, for example). In some aspects, the gas inlet may be connected to a valve, faucet, spout, or flow control fixture (1050). The gas source may include a predetermined and/or premixed preferred gas composition. In some aspects, the gas tank 1040 may include a gas concentration comprising approximately six percent oxygen gas and approximately five percent carbon dioxide gas to result in the desired partial pressures for oxygen and carbon dioxide. It will be appreciated that other suitable concentration percentages can be utilized and varied based on the desired values that can depend on individual blood values of the neonate. In some exemplary aspects, the gas flow rate can be supplied at approximately 200 standard cubic meters per minute (SCCM), and the blood flow can be maintained at approximately 85 mL/min. The particular flow rates of the gas and/or the blood can be varied by the flow controller 1050, and other flow rates are envisioned. The gas outlet 816 may be connected to a different portable tank (not shown) or to a vent (not shown). In some aspects, the gas outlet 816 may open to ambient.

The oxygenator 800 may be disposed in line with the blood conduit 1202 so as to separate the blood conduit 1202 into at least two portions. A first blood conduit portion 1204 may extend between the first end 1208 of the blood conduit 1202 and the liquid inlet 804 of the oxygenator 800. A second blood conduit portion 1206 may extend from the liquid outlet 808 of the oxygenator 800 and the second end 1212 of the blood conduit 1202. In operation, liquid through the blood conduit 1202 may be moved from the first portion 1204 toward the second portion 1206, or, alternatively, from the second portion 1206 to the toward the first portion 1204. For purposes of this disclosure, the first portion 1204 may be referred to as the arterial portion of the blood circuit 1200, while the second portion 1206 may be referred to as the venous portion of the blood circuit 1200, but it will be appreciated that this nomenclature and functionality can be reversed, such that the first portion 1204 may be the venous portion and the second portion 1206 may be the arterial portion. It should be understood that in such a reversed arrangement, the liquid inlet 804 of the oxygenator 800 may be connected to the second portion 1206, while the liquid outlet 808 may be connected to the first portion 1204.

With continued reference to FIG. 6, the first portion 1204 of the blood conduit 1202 may be designed to be connected to an artery of the neonate's umbilical cord (not shown). The first portion 1204 may include a first end connector 1216 (alternatively referred to as an arterial connector 1216) at the first end 1208. The arterial connector 1216 may include a cannula configured to be connected to a blood vessel in the neonate (e.g., to an artery in the umbilical cord). The arterial connector 1216 may be dimensioned according to desired use and may have predetermined length, diameter, gauge size, connectors, retention members, and other preferred or required components for cannulating a blood vessel and for retaining the cannula within the blood vessel after cannulation.

In some aspects, the first end 1208 may include a plurality of arterial connectors 1216, with each of the plurality of arterial connectors 1216 being configured to be connected to separate blood vessels in the umbilical cord of the neonate. As shown in the exemplary embodiment of FIG. 6, the blood circuit 1200 may include two arterial connectors 1216 at the first end 1208. In other embodiments, the blood circuit 1200 may include 1, 2, 3, 4, or another suitable number of arterial connectors 1216 at the first end 1208. The first portion 1204 of the blood conduit 1202 may be dimensioned according to preferred sizes, for example, cannulas that are 7 French in size, 8 French, or 9) French. The inner diameter in such aspects may range from about 1/32 inch to about 1 inch, between about 1/16 inch to about 0.5 inch, or another suitable range. In some exemplary embodiments, the inner diameter may be approximately 0.133 inch. The outer diameter may be in a range of between about 2/16 inch and about 1.5 inch, between about 4/16 and about 1 inch, or another suitable range. In some exemplary embodiments, the outer diameter may be approximately 5/16 inch. In some aspects, the first portion 1204 may include larger cannula sizes, such as 14 French, 15 French, 16 French, or another suitable size. The inner diameter of at least part of the first portion 1204 may range from about 1/16 inch to about 1 inch, between about 2/16 inch and about ½ inch, or another suitable range. In some exemplary embodiments, the inner diameter may be approximately 3/16 inch. The outer diameter may range from about 2/16 inch to about 1.25 inch, between about 4/16 to about 1 inch, or another suitable range. In some exemplary embodiments, the outer diameter may be approximately 5/16 inch.

The first portion 1204 may include one or more access ports 1224 through which the interior of the blood conduit 1202 may be accessed. In some aspects, liquid may be drawn out from inside the blood conduit 1202 via one or more access ports 1224. Additionally, or alternatively, one or more liquids, solids, or solutions mixtures may be introduced into the blood conduit 1202 via the one or more access ports 1224 (e.g., heparin, nutrition, and/or the like). In some aspects, one or more sensors for measuring parameters of the priming liquid and/or the neonatal blood in the blood circuit 1200 (as will be described in detail below) may be measured through the one or more access ports 1224. The one or more access ports 1224 may be connectable to a syringe and/or a separate tubular line. The one or more access ports 1224 may be selectively opened or close to permit or preclude, respectively, liquid communication therethrough with the interior of the blood conduit 1202. The one or more access ports 1224 may include a movable valve, a Luer connector, a stopcock, a resealable membrane, a capped opening, or another suitable connector commonly utilized in the medical field.

The second portion 1206 of the blood conduit 1202 may be designed to be connected to a vein of the neonate's umbilical cord (not shown). The second portion 1206 may include a second end connector 1220 (alternatively referred to as a venous connector 1220) at the second end 1212. The second end connector 1220 may include a cannula configured to be connected to a blood vessel in the neonate (e.g., to a vein in the umbilical cord). The second end connector 1220 may be dimensioned according to desired use and may have predetermined length, diameter, gauge size, connectors, retention members, and other preferred or required components for cannulating a blood vessel and for retaining the cannula within the blood vessel after cannulation. With continued reference to FIG. 6, the second portion 1206 may include a single second end connector 1220. However, it should be appreciated that the embodiments depicted in the figures are not limiting, and that other aspects of the blood circuit 1200 may include a plurality of venous connectors 1220, such as 2, 3, 4, or another suitable number of venous connectors 1220.

The second portion 1206 of the blood conduit 1202 may be dimensioned according to preferred sizes, for example, cannulas that are 14 French, 15 French, 16 French, or another suitable size. In some aspects, the second portion 1206 may have cannulas that are larger than the cannulas of the first portion 1204. The inner diameter of at least part of the first portion 1204 may range from about 1/16 inch to about 1 inch, between about 2/16 inch and about ½ inch, or another suitable range. In some exemplary embodiments, the inner diameter may be approximately 3/16 inch. The outer diameter may range from about 2/16 inch to about 1.25 inch, between about 4/16 to about 1 inch, or another suitable range. In some exemplary embodiments, the outer diameter may be approximately 5/16 inch. Alternatively, the second portion 1206 may include relatively smaller than listed above, for example having 7 French in size, 8 French, or 9 French size cannulas, or other suitable sizes. The inner diameter in such aspects may range from about 1/16 inch to about 1 inch, between about 2/16 inch to about 0.5 inch, or another suitable range. In some exemplary embodiments, the inner diameter may be approximately 0.133 inch. The outer diameter may be in a range of between about 2/16 inch and about 1.5 inch, between about 4/16 and about 1 inch, or another suitable range. In some exemplary embodiments, the outer diameter may be approximately 5/16 inch.

The second portion 1206 may include one or more access ports 1224. Unless noted otherwise, the one or more access ports 1224 may be substantially similar to, or the same as, the one or more access ports 1224 as described above with relation to the first portion 1204. It should be appreciated that the nomenclature regarding the first end connector 1216 and the second end connector 1220 may depend on the particular use and operation of the blood circuit 1200. For example, if liquid is flown from the first end 1208 towards the second end 1212, the first end connector 1216 may be the arterial connector and the second end connector 1220 may be the venous connector; conversely, if the liquid is flown in a direction from the second end 1212 toward the first end 1208, the first end connector 1216 may be referred to as the venous connector, and the second end connector 1220 may be referred to as the arterial connector.

### Connection Between Blood Circuit and Priming Circuit and Priming Process

Prior to connecting the blood circuit 1200 to the neonate, it may be advantageous to prime the blood circuit 1200. Priming can include introducing one or more liquids into the blood circuit 1200. This may be advantageous to immediately supply the neonate with the desired priming liquid that can include nutrients and/or blood thinners. The priming process may also serve to adjust the physical and/or chemical parameters of the priming liquid introduced into the blood circuit 1200. Adjustable parameters may include temperature, pressure, pH, osmolarity, compositions, gas saturations, and other characteristics of priming liquid. Ensuring that the priming liquid introduced into the neonate has the desired parameters can decrease changes of injury to the neonate due to exceedingly hot or cold priming liquid or improper concentrations of gases that are not optimal for neonatal development. Priming the blood circuit 1200 may also serve to remove undesirable particulates or gases from the priming circuit 1300 prior to connecting the priming circuit 1300 to the neonate. For example, introducing the priming liquid into the blood circuit 1200 may displace and remove therefrom any trapped air bubbles that could otherwise enter the neonatal bloodstream if not removed prior to cannulation. This process may also serve to flush out any particulates, debris, or other unwanted components inside the blood circuit 1200. Generally, the purpose of priming the blood circuit 1200 prior to connecting the blood circuit 1200 to the neonate is to ensure that when the neonate is cannulated and placed in liquid communication with the blood circuit 1200 that the neonate is in an optimal environment as close as possible to the physiological conditions of a natural womb.

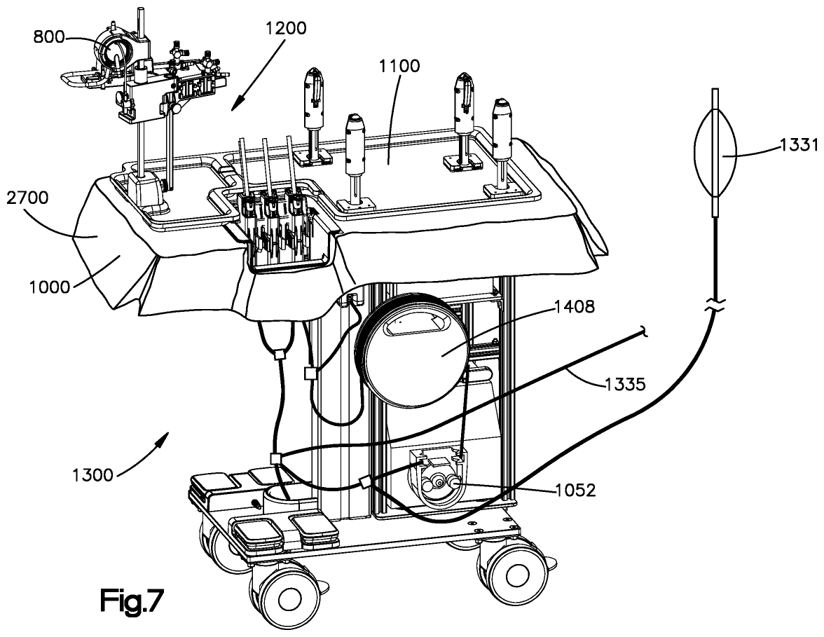

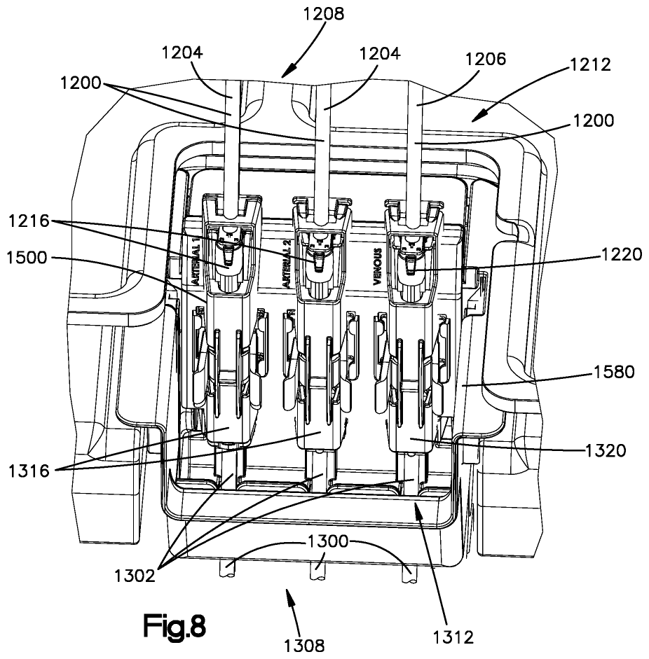

To prime the blood circuit 1200, the blood circuit 1200 may be connected to the priming circuit 1300. Referring to FIGS. 7 and 8, an exemplary connection is depicted, showing a blood circuit 1200 in fluid communication with the priming circuit 1300. The priming circuit 1300 may be affixed to the cart 1000 and operably connected to, or disposed adjacent, the pump 1052 and the heater 1048 as was described above. A priming supply source 1331 and a waste destination 1335 can be connected to the priming circuit 1300. The blood circuit 1200 is depicted on the cart 1000 (shown on the tray 1100, which will be described further below).

FIG. 8 shows a closeup connection interface between the blood circuit 1200 and the priming circuit 1300. The blood conduit 1202 and the priming conduit 1302 may be connected to each other via a connection assembly 1500 and may be arranged in a connection cradle 1580 (described further below). The first end 1208 of the blood circuit 1200 may be connected to the first end 1308 of the priming circuit 1300. Similarly, the second end 1212 of the blood circuit 1200 may be connected to the second end 1312 of the priming circuit 1300. The first end connector 1216 of the blood circuit 1200 may releasably connect with the first end connector 1316 of the priming circuit 1300. The second end connector 1220 of the blood circuit 1200 may releasably connect with the second end connector 1320 of the priming circuit 1300. In embodiments where the blood circuit 1200 includes a plurality of first end connectors 1216, the priming circuit 1300 may include a corresponding equal number of first end connectors 1316, with each of the first end connectors 1216 of the blood circuit 1200 being connectable to a different first end connector 1316 of the priming circuit. Similarly, in embodiments where the blood circuit 1200 includes a plurality of second end connectors 1220, the priming circuit 1300 may include a corresponding equal number of second end connectors 1320, with each of the second end connectors 1220 of the blood circuit 1200 being connectable to a different second end connector 1320 of the priming circuit.

When the first end 1208 of the blood circuit 1200 is connected to the first end 1308 of the priming circuit 1300 and the second end 1212 of the blood circuit 1200 is connected to the second end 1312 of the priming circuit 1300, a looped circuit is established that includes both the priming circuit 1300 and the blood circuit 1200. That is, the priming circuit 1300 is in liquid communication with the blood circuit 1200, and liquid (e.g., priming liquid) can be circulated through the entirety of the priming circuit 1300, moved from the priming circuit 1300 to the blood circuit 1200, circulated through the entirety of the blood circuit 1200, and moved back to the priming circuit 1300. This arrangement allows the blood circuit 1300 to be primed with the desired priming liquid. Components that are operably connected to and are in liquid communication with the blood circuit 1200 and/or the priming circuit 1300 may also be primed in this arrangement (e.g., the oxygenator 800). The sweep gas can be introduced when priming the blood circuit 1200.

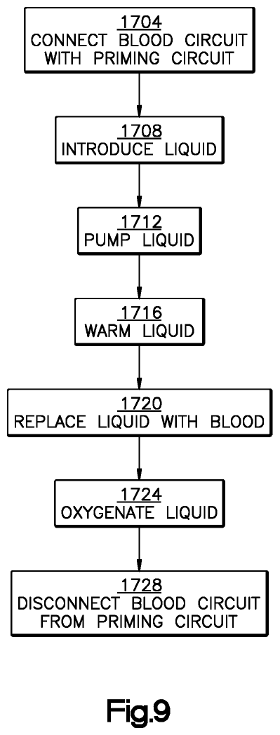

An exemplary priming method 1700 is depicted in the flow chart shown in FIG. 9. Prior to beginning to prime the blood circuit 1200 and its components, and as shown in step 1704, the blood circuit 1200 can be connected to the priming circuit 1300 to form the interconnected loop circuit between the priming and blood circuits 1300 and 1200 as described above.

In operation, the priming circuit 1300 may receive the priming liquid from the priming liquid supply source 1331. In step 1708, to begin the priming process, the priming liquid supply source 1331 is connected to the priming circuit 1300. Alternatively, if the priming liquid supply source 1331 is already connected, the priming liquid may be introduced into the priming liquid supply source 1331 for priming. The priming liquid can travel from the source 1331 along the supply line 1330) and into the priming conduit 1302 through the supply connector 1328. The supply connector 1328 should be in the open configuration at this time to allow the priming liquid to flow therethrough into the priming conduit 1302. If the supply connector 1328 is in the closed configuration, the supply connector 1328 can be moved into the open configuration during this step.

In step 1712, the priming liquid is flown through the priming circuit 1300. The pump 1052 that is operably connected to the priming circuit 1300 can be actuated to cause movement of the priming liquid through the priming conduit 1302 towards the blood circuit 1200. The actuation of the pump 1052 can cause an increase in pressure within the priming conduit 1302 that forces the fluid (including priming liquid or air) in the priming conduit 1302 to move along the priming conduit 1302 in the desired direction. In some aspects, the pump 1052 may be configured to cause the priming liquid to be moved towards the first end 1308 of the priming conduit 1302, such that the priming liquid is discharged out of the one or more first end connectors 1316 into the one or more connected first end connectors 1216 of the first end 1208 of the blood conduit 1202. In such aspects, the actuation of the pump 1052 causes the priming liquid to continue to flow through the blood circuit 1200 from the first end 1208 towards the second end 1212, and then to be discharged out of the second end 1212 through the one or more second end connectors 1220 back into the priming circuit 1300 through the one or more connected second end connectors 1320 at the second end 1312 of the priming conduit 1302. In some aspects, the pump 1052 may be configured to cause movement of the priming liquid in the opposite direction than described above. In such aspects, the pump 1052 may be configured to cause the priming liquid to be moved towards the second end 1312 of the priming conduit 1302, such that the priming liquid is discharged out of the one or more second end connectors 1320 into the one or more connected second end connectors 1220 of the blood conduit 1202. In such aspects, the actuation of the pump 1052 causes the priming liquid to continue to flow through the blood circuit 1200 from the second end 1212 towards the first end 1208, and then to be discharged out of the first end 1208 through the one or more first end connectors 1216 back into the priming circuit 1300 through the one or more connected first end connectors 1316 at the first end 1308 of the priming conduit 1302. In some aspects, the liquid is circulated back to the source container 1331 to capture any air bubbles in the source container 1331.

During the priming process, while the priming liquid is being circulated within the priming circuit 1300 and the blood circuit 1200, the parameters of the priming liquid may be adjusted until they adhere to the desired targets or thresholds. In some aspects, as shown in step 1720, the temperature of the priming liquid may be raised to a predetermined threshold and then maintained within the desired predetermined temperature range. The temperature of the priming liquid can be monitored by the temperature sensor 1324 as described above. The priming liquid can be heated by the heater 1048 to raise the temperature. The heater 1048 may be adjusted to increase or decrease heat output to respectively increase or decrease heat being applied to the priming liquid. In some aspects, the desired temperature range may be similar to physiological womb temperature, for example in the range of between about 35 degrees Celsius and about 40 degrees Celsius, or more specifically between about 36 degrees Celsius and about 38 degrees Celsius. Other suitable temperature thresholds are also envisioned.

In some aspects, the pump 1052 may be reversible, such that the priming liquid may be pumped in either of the directions described above. In some exemplary aspects, the priming liquid is moved from the priming liquid source 1331 into the priming conduit 1302 and towards the second portion 1206 of the blood conduit 1200, which may correspond to the venous connection(s) of the blood circuit 1200. This way, the priming liquid receives heat from the heater 1048 and is moved into the blood circuit 1200 while heated, and as the priming liquid is moved through the blood circuit 1200 and back into the priming circuit 1300, the priming liquid gradually loses heat. After the priming process, when the primed blood circuit 1200 is disconnected from the priming circuit 1300, the warmest blood will be adjacent the second end 1206 (i.e., the venous connector). Thus, when the second end 1206 is connected to the umbilical cord (i.e., to a vein of the umbilical cord), the vein of the umbilical cord will receive the warmest blood from the blood circuit 1200, which may be advantageous to minimize risk of venous spasms that can arise due to undesirably low blood temperature.

When a desired volume of priming liquid has been introduced into the priming circuit 1300, the priming liquid supply source 1331 may be taken out of liquid communication from the priming circuit 1300. This can be done by moving the supply connector 1328 to the closed configuration, such that liquid flow is precluded from the supply line 1330 into the priming conduit 1302. In some aspects, the desired priming liquid may be substantially equal to the total aggregate volume of the priming circuit 1300 and the blood circuit 1200. In some aspects, the desired volume of priming liquid may be greater than the total aggregate volume of the priming circuit 1300 and the blood circuit 1200, such that a portion of the desired volume of the priming liquid is removed from the loop circuit formed by the connected priming circuit 1300 and the blood circuit 1200. This allows for unwanted fluids or particulates within the priming circuit 1300, the blood circuit 1200, or any of the connected components to be flushed out of the connected priming and blood circuits 1300 and 1200. In step 1720, a portion of the priming liquid can be flushed from the priming circuit 1300. To flush out a portion of the priming liquid, the waste connector 1332 may be moved into the open configuration to allow flow of the priming liquid from the priming conduit 1302 into the connected waste line 1334. From the waste line 1334, the priming liquid can be moved toward and into the waste receptacle 1335. When the desired amount of priming liquid has been removed from the priming conduit 1302, the waste connector 1328 may be moved into the closed position such that no more priming liquid can flow therethrough into the waste line 1334. The priming liquid can be replaced by blood.

Once the initial priming liquid has been replaced by blood, the dissolved gas concentrations of the priming liquid can also be adjusted. As depicted by step 1724, as the priming liquid is moved through the oxygenator 800, one or more gases can be introduced into the priming liquid, while one or more gases can be removed therefrom via the oxygenator. In some aspects, the sweep flow rate and composition through the oxygenator 800 may control the partial pressure of oxygen gas and carbon dioxide gas in the blood. A purpose of the gas exchange is to provide the priming liquid with gas partial pressures that are similar to those in naturally occurring physiological environments (e.g., when the neonate is in the womb). In some aspects, the desired target range of partial pressure of oxygen gas in the priming liquid may be between about 30 mmHg and about 50 mmHg, and more specifically between about 35 mmHg and about 40 mmHg. The partial pressure of carbon dioxide gas in the priming liquid may be between about 30 mmHg and about 50 mmHg, and more specifically between about 35 mmHg and about 40 mmHg. Nitrogen gas may also be added or removed for balance. The gas exchange process can additionally help reach and maintain a target pH concentration of the priming liquid. In some aspects, the preferred pH range of the priming liquid may be between about 7 and about 8, and more specifically between about 7.2 and about 7.6. In some exemplary aspects, the target pH range may be between about 7.35 and about 7.4. The gas being utilized can be supplied by the console or stored in a prepared gas tank 1040 with the desired concentration of gases therein. Varying levels of oxygen gas, carbon dioxide gas, and nitrogen gas can be utilized to achieve varying physiological blood values that correspond to the neonate's blood gas levels.

In some aspects, a plurality of priming liquids may be used. As such, the priming circuit 1300 and/or the blood circuit 1200 may first be primed as described above with a first priming liquid, and then the priming circuit 1300 and/or the blood circuit 1200 may be primed with a second priming liquid. The first and second priming liquids may be the same, or, alternatively, the first and second priming liquids may be different or may include different components therein. In some specific embodiments, the first priming liquid may include a synthetic liquid (e.g., a crystalloid solution with desired electrolytes, such as PlasmaLyte), and the second priming liquid may include conditioned donor blood. If multiple priming liquids are used, the multiple priming liquids may be connected to and introduced to the priming circuit 1300 either simultaneously or one after the other. In some aspects, the first priming liquid may be introduced into the priming liquid supply source 1331 first, and then the second priming liquid may be introduced into the priming liquid supply source 1331. In alternate embodiments, each priming liquid may be disposed at its own respective priming liquid supply 1331, and each priming liquid supply source 1331 may be selectively placed into and out of liquid communication with the priming conduit 1302 in the desired order and such that the desired predetermined amount of each of the first and second priming liquids can be introduced into the priming circuit 1300.

When the blood circuit 1200 and any other desired components have been sufficiently primed, the blood circuit 1200 may be disconnected from the priming circuit 1300 in step 1728. After the blood circuit 1200 is primed and separated from the priming circuit 1300, the blood circuit 1200 may then be connected to the neonate via umbilical cord blood vessel cannulation.

It will be appreciated that additional steps in the priming process may be envisioned, and that the above listing of steps is exemplary and not limiting. Unless noted otherwise, the steps described above may be performed in any order relative to each other. For example, the priming liquid may be heated either before, during, or after being oxygenated or in any combination. In some aspects, portions of the priming liquid may be heated at the same time that other portions of the priming liquid are being oxygenated. One or more of the steps described above may be performed a plurality of times, and may be repeated sequentially or with other steps between repeated iterations. One or more of the steps described above may be performed simultaneously in some embodiments.

### Connection Assembly

As briefly noted above, the priming circuit 1300 may be releasably connected to the blood circuit 1200 via a connection assembly 1500 (shown generally in FIG. 8). The connection assembly 1500 serves to provide an interface between each end of the blood and priming conduits 1202 and 1302 to place the blood circuit 1200 into liquid communication with the priming circuit 1300, such that the priming liquid can be circulated between the priming and blood circuits 1300 and 1200. The first end 1208 of the blood conduit 1202 can be connected to the first end 1308 of the priming conduit 1302, and the second end 1212 of the blood conduit 1202 can be connected to the second end 1312 of the priming conduit 1302. In some aspects, the connection assembly 1500 may releasably engage the one or more first end connectors 1216 with respective one or more first end connectors 1316 (see, e.g., FIG. 8). Similarly, the connection assembly 1500 may releasably engage the one or more second end connectors 1220 with the respective one or more second end connectors 1320. The connection assembly 1500 may be actuated based on relative positioning of one or more components therein (as will be detailed below). The engagement and disengagement of the connectors of the priming circuit 1300 and the blood circuit 1200 may be spring-loaded or otherwise biased or pre-tensed, such that the connection is not permanent and is not established for long durations. That is, the interface between the priming and blood circuits 1300 and 1200 may be severed so that the respective connectors are not in contact with each other until the desired time (for example, until immediately prior to the priming process). This allows for the connection components to be protected during shipping, sterilization, and/or storage, which can decrease risk of compromising the integrity of the components.

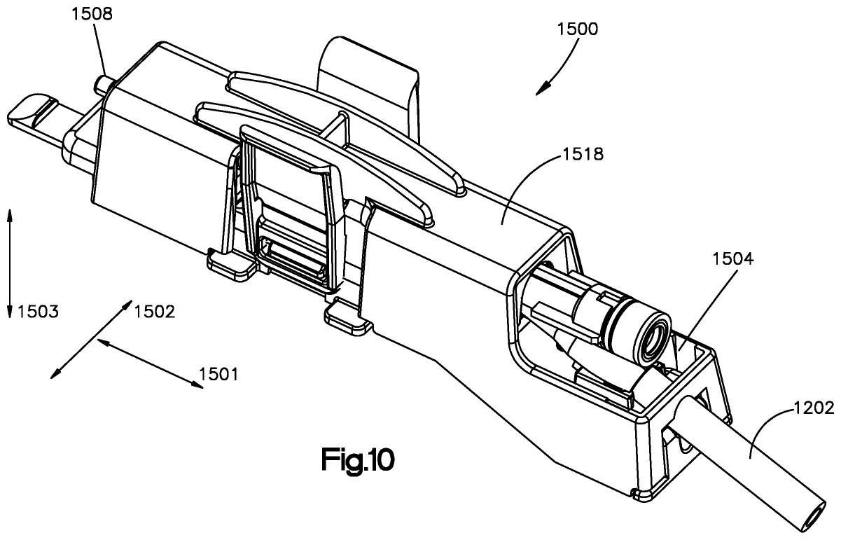

Referring to FIGS. 10-13, exemplary embodiments of connection assemblies 1500 are depicted. It will be appreciated that other suitable connection mechanisms can be utilized to connect the priming circuit 1300 with the blood circuit 1200, and that similar systems can be operated without the disclosed connection assemblies 1500. A connection assembly 1500 includes a body 1504 that includes thereon at least two connectors for receiving the priming conduit 1302 and the blood conduit 1202 and for forming an engagement between the blood and priming conduits 1202 and 1302. The connection assembly 1500 may include a cover 1518 configured to at least partly enclose the body 1504 and the connectors to protect the components of the connection assembly 1500 from debris and/or damage. Components of the connection assembly 1500, as well as the respective connectors from the blood conduit 1202 and the priming conduit 1302, can also be protected from non-sterile surroundings during use. The cover 1518 may be opaque or transparent. The cover 1518 may be movable between a closed configuration, in which the cover 1518 contacts the body 1504 and obstructs access to the components defined or attached to the body 1504, and an open configuration, in which the cover 1518 is spaced from the body 1504 such that a user can access the components on or in the body 1504. In some aspects, the cover 1518 may be fully separated from the body 1504 when the cover 1518 is moved to the open configuration, or, alternatively, in other aspects, the cover 1518 may remain attached to the body 1504 when moved to the open configuration (for example, via a hinged connection).

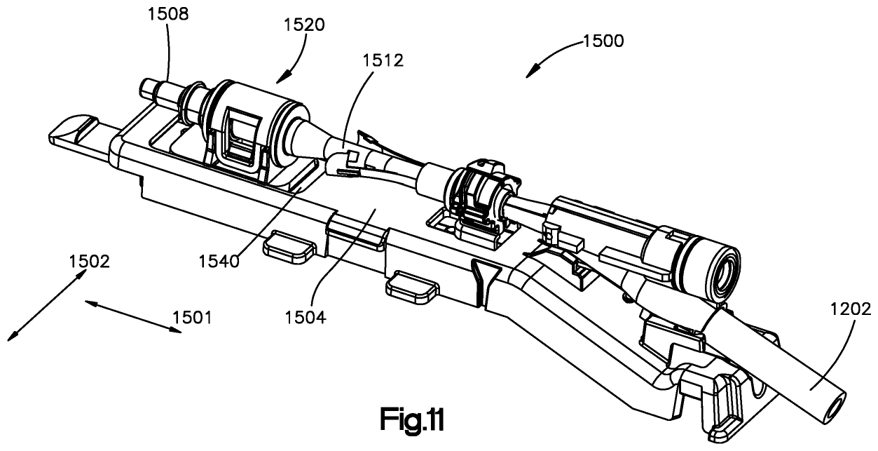

With further reference to FIGS. 11-13, a blood circuit connector 1512 can be defined on, or attached to, the body 1504. The blood circuit connector 1512 is configured to receive the blood conduit 1202 thereon and to liquidly communication with the blood circuit 1200. A priming circuit connector 1508 is disposed on, or attached to, the body 1504 and may be spaced from the blood circuit connector 1512 along an axial direction 1501. The priming circuit connector 1508 is configured to receive the priming conduit 1302 thereon and to liquidly communicate with the priming circuit 1300. When the blood conduit 1202 is fully engaged with the blood circuit connector 1512 and the priming conduit 1302 is fully engaged with the priming circuit connector 1508, a liquid flow path can be established between the priming conduit 1302 and the blood conduit 1202.

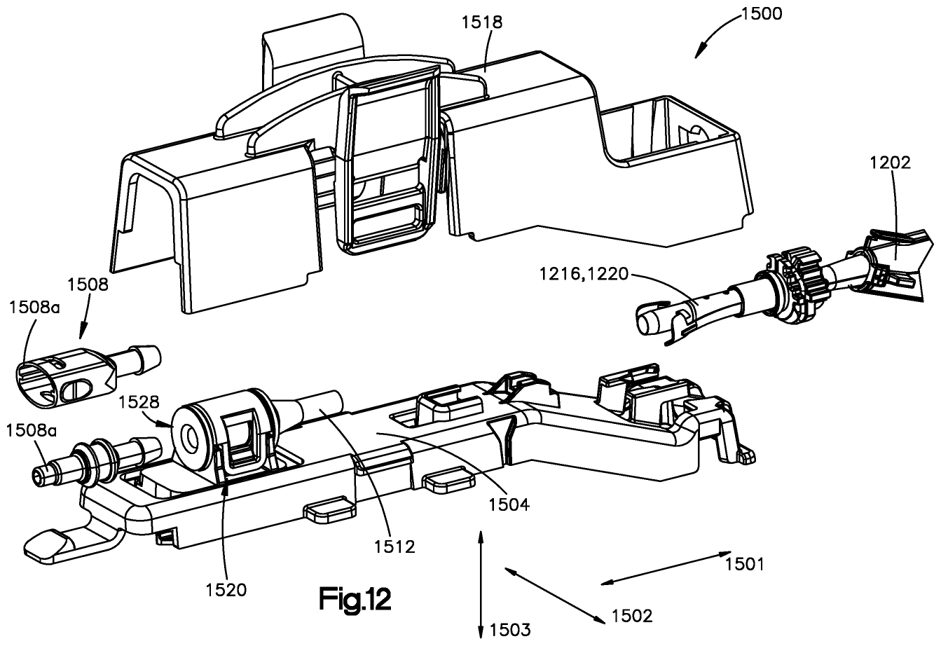

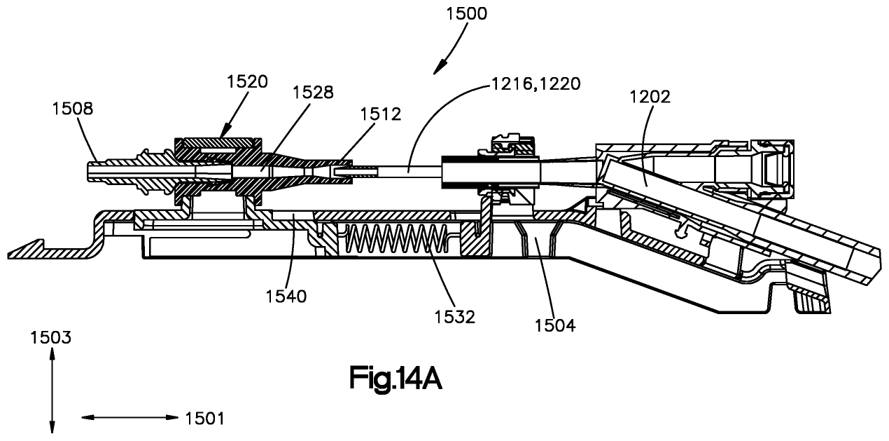

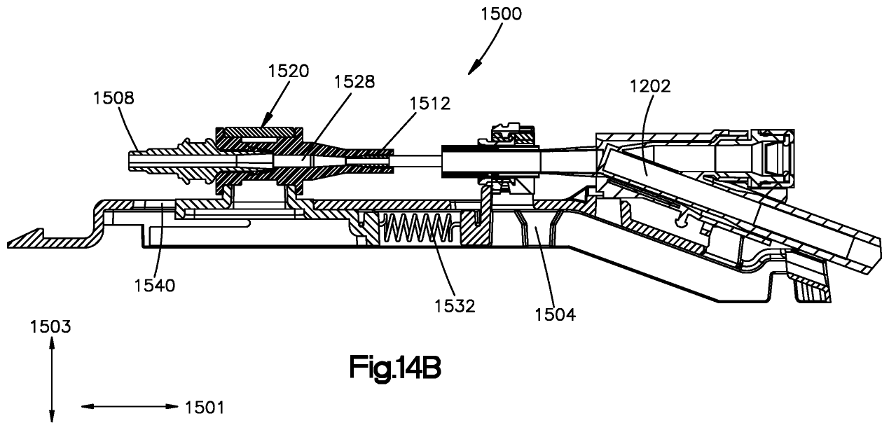

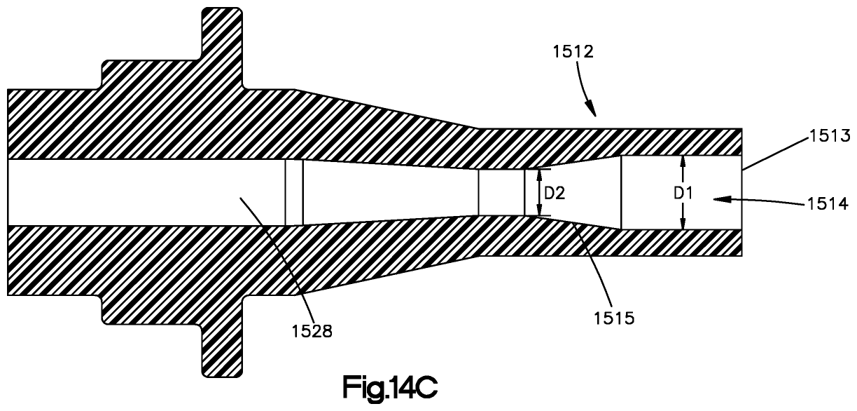

In some aspects, the priming circuit connector 1508 and the blood circuit connector 1512 may be disposed on a carriage member 1520 that is disposed on or in the body 1504. The priming circuit connector 1508 may be spaced away from the blood circuit connector 1512 along the axial direction 1501. A chamber 1528 may be defined in the carriage 1520 between the priming circuit connector 1508 and the blood circuit connector 1512 (labeled in FIGS. 12, 14A, 14B). The chamber 1528 may receive the priming liquid therein when the priming circuit 1300 is connected to the blood circuit 1200. The chamber 1528 may define a flow path between the priming conduit 1302 and the blood conduit 1202.

In some aspects, the connection assembly 1500 may be configured to have multiple connection states or configurations. In a full connection configuration, the blood circuit 1200 and the priming circuit 1300 are in liquid communication with each other and are connected via the liquid flow path extending through the chamber 1528 between the priming circuit connector 1508 and the blood circuit connector 1512. In the full connection configuration, the chamber 1528 may be substantially sealed such that priming liquid can move into the chamber 1528 only from the priming conduit 1302 and out of the chamber 1528 into the blood conduit 1202, or vice versa. That is, in the full connection configuration, the chamber 1528 and the liquid flow path extending therethrough is sealed so that substantially all of the liquid that flows into the chamber 1528 from one of the priming conduit 1302 and the blood conduit 1202 is moved into the other of the priming conduit 1302 and the blood conduit 1202 (i.e., there is no leakage between the priming conduit 1302 and the priming circuit connector 1508 or between the blood conduit 1202 and the blood circuit connector 1512.

In a partial connection configuration, the blood circuit 1200 and the priming circuit 1300 may be in liquid communication with each other and connected via the liquid flow path extending through the chamber 1528, but the connection may not be liquid-tight. In some aspects, in the partial connection configuration, the blood conduit 1202 may be in contact with the blood circuit connector 1512, but a gap may be defined between the blood conduit 1202 and the blood circuit connector 1512. Such an arrangement allows the blood circuit connector 1512 and the blood conduit 1202 to be axially aligned to facilitate movement into the full connection configuration, while maintaining a separation of connection points. Maintaining separation of connections may help preclude damage to the connector components and may decrease risk of contamination or bacterial growth. In the partial connection configuration, the blood conduit 1202 and the priming conduit 1302 are arranged relative to each other such that they can be readily moved into the fully connected configuration by a user without having to spend excessive time aligning components (relative to a fully disconnected configuration, in which the priming conduit 1302 is fully separate from the priming circuit connector 1508, the blood conduit 1202 is fully separate from the blood circuit connector 1512, or both).

To transition between the partially connected configuration to the fully connected configuration, a portion of the connection assembly 1500 may be moved along the axial direction 1501 such that a liquid-tight connection is established between the priming conduit 1302 and the priming circuit connector 1508, between the blood conduit 1202 and the blood circuit connector 1512, or both. Referring to FIGS. 10-13, the carriage member 1520) may be movable relative to the body 1504 along the axial direction 1501. The body may define a recess 1540 that is configured to receive the carriage 1520 therein. The carriage 1520 may have a first position within the recess 1540 relative to the body 1504 (see, e.g., FIG. 14A) and a second position within the recess 1540 axially spaced from the first position along the axial direction 1501 (see, e.g., FIG. 14B). When the priming conduit 1302 is connected to the priming circuit connector 1508, the blood conduit 1202 is connected to the blood circuit connector 1512, and the carriage 1520 is in the first position, the connection assembly 1500 may be in the partially connected configuration. When the priming conduit 1302 is connected to the priming circuit connector 1508, the blood conduit 1202 is connected to the blood circuit connector 1512, and the carriage 1520 is in the second position, the connection assembly 1500 may be in the fully connected configuration.

The carriage 1520 may be moved between the first and second positions by applying a force along the axial direction 1501 to the carriage 1520. In some aspects, the carriage 1520 may be moved from the first position to the second position by an actuator 1532. The actuator 1532 can be configured to apply a force to the carriage 1520 to cause the carriage 1520) to slidably move relative to the body 1504 along the axial direction 1501. In some aspects, the actuator 1532 may include a resilient spring. The resilient spring may apply a tensile force on the carriage 1520 to cause the carriage 1520 to move. Alternatively, the resilient spring may apply a compressive force on the carriage 1520. In some aspects, the actuator 1524 may include a deformable member that is configured to be placed in contact with the carriage 1520 and is biased towards causing movement of the carriage 1520 along the axial direction 1501. In the exemplary embodiments depicted in FIGS. 10-14B, the connection assembly 1500 is shown having a spring actuator 1532 that is held in tension between the body 1504 and the carriage 1520. In such embodiments, the spring actuator 1532 can apply a tensile force onto the carriage 1520 to pull the carriage 1520 along the axial direction 1501 relative to the body 1504 to transition the connection assembly 1500 from the partially connected configuration to the fully connected configuration. Referring specifically to FIGS. 14A and 14B, the connection assembly 1500 is shown in the partially connected configuration in FIG. 14A and in a fully connected configuration in FIG. 14B. In FIG. 14A, the end connector of the blood conduit 1202 (which can be either the first end connector 1216 or the second end connector 1220) is axially aligned with, and is partially inserted into, the blood circuit connector 1512. However, the end connector is not fully inserted into the blood circuit connector 1512, and thus a liquid-tight seal between the blood conduit 1202 and the chamber 1528 is not established in this configuration. When the spring actuator 1532 causes the carriage 1520 to move along the axial direction 1501 towards the blood conduit 1202, as shown in FIG. 14B, the end connector of the blood conduit 1202 (e.g., the first end connector 1216 or the second end connector 1220) is axially aligned with, and is fully inserted into, the blood circuit connector 1512. When the end connector is fully inserted into the blood circuit connector 1512, a liquid-tight seal is established between the end connector and the blood circuit connector 1512, and the blood conduit 1202 is in liquid communication with the chamber 1528. The connection assembly 1500 is in the fully connected configuration. It will be understood that although a priming conduit 1302 is not depicted in FIGS. 14A and 14B, for the connections to be established, and for the connection assembly 1500 to be in the fully connected configuration, the priming conduit 1302 should be fully connected to the priming circuit connector 1508 as described above. In the fully connected configuration, liquid-tight seals are defined between the priming conduit 1302 and the chamber 1528, as well as between the blood conduit 1202 and the chamber 1528.

Referring to FIG. 14C, an exemplary blood circuit connector 1512 is depicted that can be utilized with one or more embodiments of connection assemblies 1500 disclosed herein. The blood circuit connector 1512 can define an opening 1513 configured to receive the blood conduit 1202 therein (e.g., a first end connector 1216 or a second end connector 1220). A channel 1514 can be defined in the blood circuit connector 1512 extending from the opening 1513 along the axial direction 1501 towards the chamber 1528. The channel 1514 may be in liquid communication with the chamber 1528. The blood circuit connector 1512 may define a constriction 1515 therein disposed between the opening 1513 and the chamber 1528. The channel 1514 may be tapered along at least a portion of its length along the axial direction 1501, such that a cross-sectional dimension of the channel 1514 decreases between the opening 1513 and the constriction 1515. The channel 1514 may have a first diameter D1 adjacent the opening 1513 and a second diameter D2 adjacent the constriction 1515. In some aspects, the first diameter D1 may be greater than the second diameter D2. It should be appreciated that the first diameter D1 should be dimensioned such that a suitable end connector of the blood conduit 1202 can be inserted therethrough into the channel 1514. In operation, when the blood conduit 1202 is not within the channel 1514, the connection assembly 1500 may be in a fully separated configuration, i.e., in neither a fully connected configuration nor a partially connected configuration. When a portion of the blood conduit 1202 is within the channel 1514 but is spaced from the constriction 1515 and does not contact the constriction 1515, the connection assembly 1500 may be in the partially connected configuration. When the portion of the blood conduit 1202 is within the channel 1514 and is in contact with the constriction 1515, the connection assembly 1500 may be in the fully connected configuration. As the blood conduit 1202 is further moved through the channel 1514 along the axial direction 1501 towards the constriction 1515, the blood conduit 1202 can contact the calls of the channel 1514 as the channel 1514 tapers. The contact between the walls of the channel 1514 and the blood conduit 1202 can define a liquid-tight seal, such that liquid can be moved from the chamber 1528 into the blood conduit 1202 without being permitted to flow around the blood conduit 1202 and leak out of the opening 1513 of the blood circuit connector 1512.

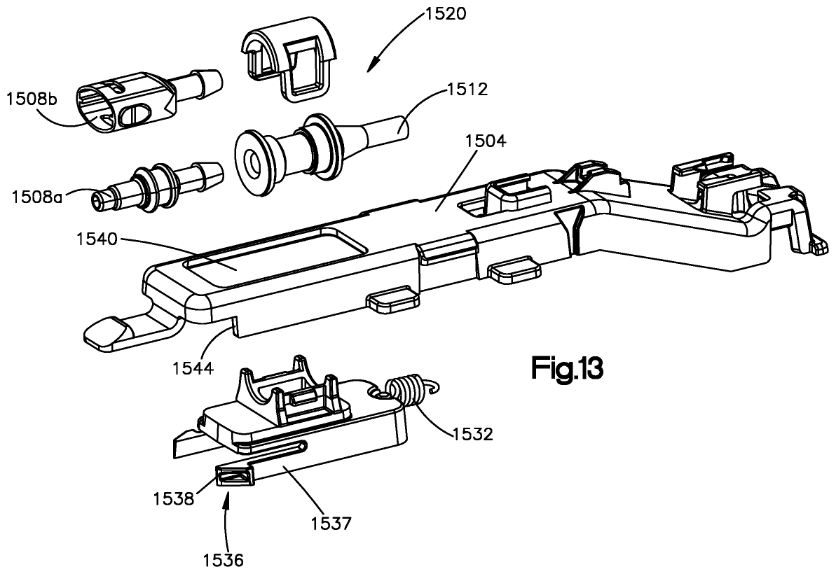

The connection assembly 1500 may be configured to be actuated between the partially connected configuration and the fully connected configuration by causing the actuator 1532 to move the carriage 1520 within the recess 1540 of the body 1504. For purposes of this disclosure, when the carriage 1520 is in the first position, the connection assembly 1500 may be in the partially connected configuration, and when the carriage 1520 is in the second position, the connection assembly 1500 may be in the fully connected configuration, although it will be appreciated that the nomenclature can be reversed in other embodiments. In some aspects, the carriage 1520) may be biased towards movement towards the second position. In such embodiments, the carriage 1520 may be physically restrained in its first position from moving into its second position. It should be understood that the restraining mechanism should be sufficient to apply the necessary force to counteract the biasing force being applied to the carriage 1520 by the actuator 1532. To move the carriage 1520 to the second position, the retraining mechanism can be removed or reduced, such that the force applied by the actuator 1532 is sufficient to cause the carriage 1520 to move to the second position. Referring again to FIG. 13, the actuator 1532 may be a spring 1532 that is operably connected to the carriage 1520 and to the body 1504. The spring 1532 may apply a tensile force on the carriage 1520 to bias the carriage 1520 towards movement along the axial direction 1501 within the recess 1540 toward the blood circuit 1200. The carriage 1520 may include a retention and release mechanism 1536. The retention mechanism 1536 can retain the carriage 1520 in its first position. The retention mechanism 1536 can be designed to counteract the biasing force being applied by the spring 1532. The retention mechanism 1536 may be any suitable retention device configured to apply a force that is opposite and at least as strong as the biasing force being applied by the spring 1532. In some aspects, the retention mechanism 1536 may include a flexible arm 1537 extending from the carriage 1520. The flexible arm 1537 may be configured to deflect in at least one direction that is orthogonal to the axial direction 1501. A projection 1538 may be defined on the flexible arm 1537. The projection 1538 may be configured to releasably contact a corresponding retention surface. As shown in FIG. 13, the body 1504 can define a shoulder 1544 thereon configured to receive the projection 1538. When the carriage 1520 is in the first position, the spring 1532 can be tensioned and applying a biasing force on the carriage 1520 towards the second position. In the first position, the projection 1538 may be in contact with the shoulder 1544 of the body 1504. The projection 1538 may be axially aligned with the shoulder 1544 along the axial direction 1501. The biasing force applied by the spring 1532 is not sufficient to overcome the engagement between the projection 1538 and the shoulder 1544, and thus the carriage 1520 is retained in the first position notwithstanding the applied tension force by the spring 1532. To move the carriage 1520 from the first position not the second position, the retention mechanism 1536 can be actuated to no longer obstruct, or to obstruct less, the biasing force applied by the spring 1532. The projection 1538 may be moved out of axial alignment with the shoulder 1544 by deflecting the flexible arm 1537 along a direction that is offset from the axial direction 1501. When the projection 1538 is not in contact with the shoulder 1544, the biasing force applied by the spring 1532 can cause the carriage 1520 to move to the second position along the axial direction 1501. In some aspects, the retention mechanism 1536 may include a plurality of flexible arms 1537, projections 1538, and respective shoulders 1544.

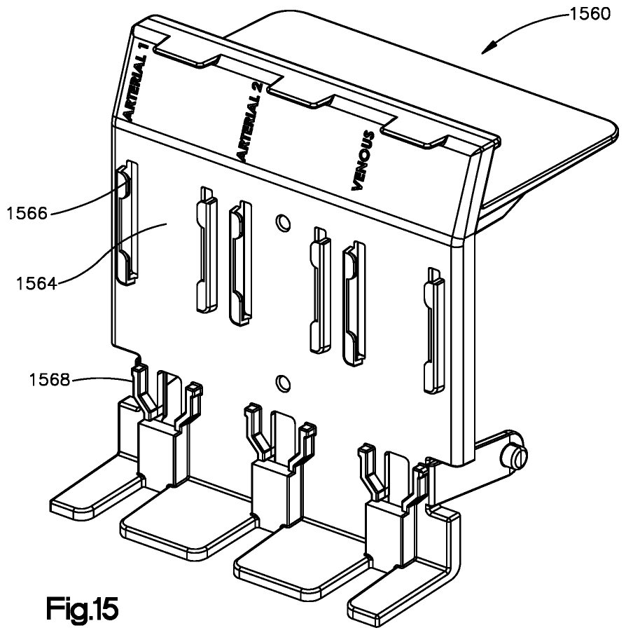

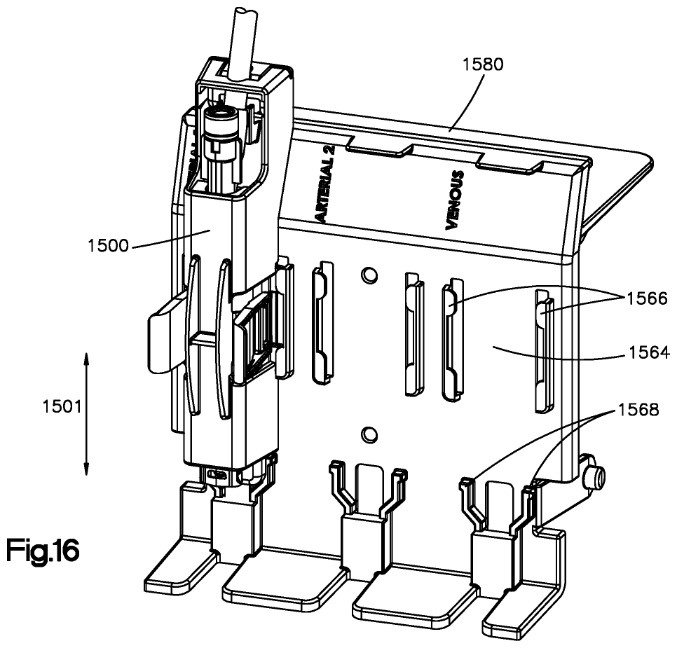

The retention mechanism 1536 may be released by a user by moving the one or more projections 1538 out of axial alignment with the one or more respective shoulders 1544. In some aspects, the retention mechanism 1536 may be released due to movement of the connection assembly 1500 and/or engagement of the connection assembly 1500 relative to or with another component. Referring to FIGS. 15 and 16, a manifold 1560 is depicted that is configured to receive thereon one or more connection assemblies 1500. The manifold 1560 can be configured to retain the connection assemblies 1500 during the priming processes. In some aspects, manifold 1560 may be used to transition each connection assembly 1500 from the partially connected configuration to the fully connected configuration (e.g., by causing movement of the carriage 1520 from the first position to the second position). The manifold 1560 includes a receiving surface 1564 configured to receive each connection assembly 1500 thereon. One or more retention channels 1566 may be defined on the receiving surface 1564 for contacting the attached connection assemblies 1500 and to releasably secure the connection assemblies 1500 to the manifold 1560.

In some aspects, the relative movement of the connection assembly 1500 along the receiving surface 1564, as well as, or alternatively, the contact between the connection assembly 1500 and the retention channels 1566 may release the retention mechanism 1536 described above to cause movement of the carriage 1520. In some aspects, the manifold 1560 may include one or more fingers 1568 defined thereon and configured to operatively contact the retention mechanism 1536 on the connection assembly 1500. The fingers 1568 may be arranged such that when the connection assembly 1500 is secured to the receiving surface 1564 within the one or more retention channels 1566, the fingers 1568 cause the projection 1538 to be moved out of axial alignment with the shoulder 1544. As described above, when the projection 1538 is not axially aligned with the shoulder 1544, the carriage 1520 may be moved to the second position due to the force applied on the carriage 1520 by the spring 1532 (or by another suitable actuator).

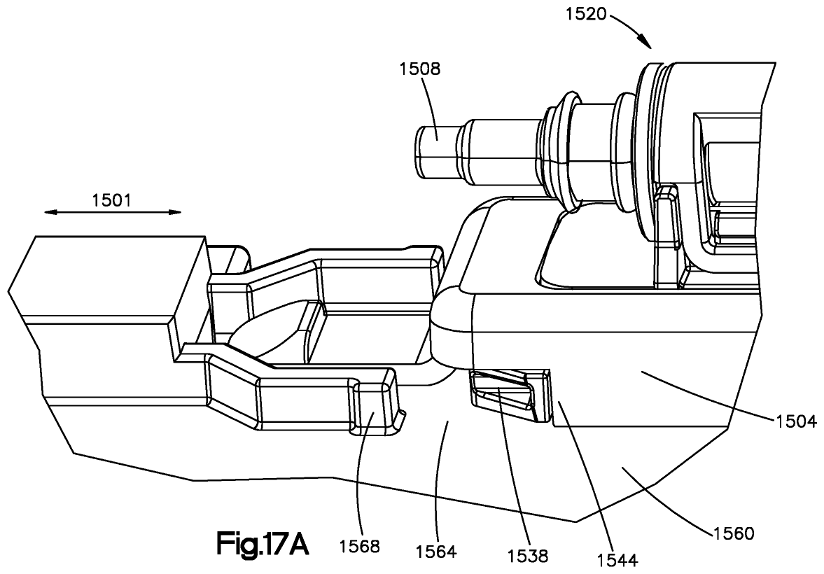

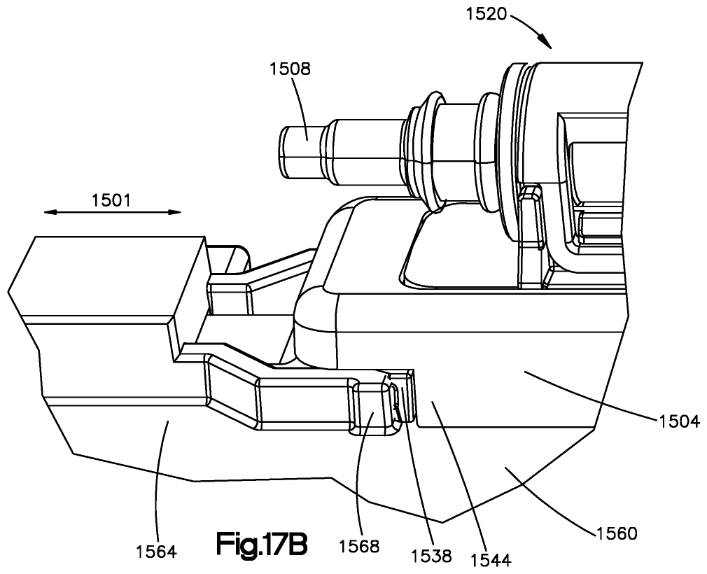

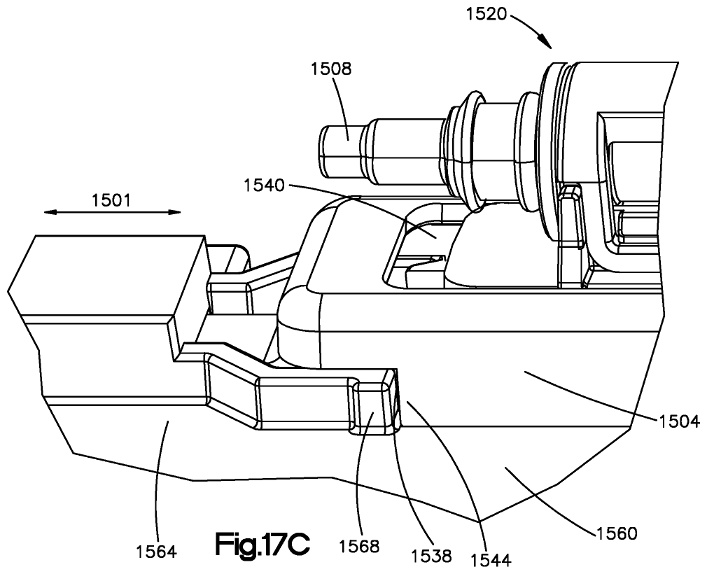

An exemplary release process is depicted in FIGS. 17A, 17B, and 17C. In FIG. 17A, a connection assembly 1500 is shown being received on the receiving surface 1564 of the manifold 1560 along the axial direction 1501. The projections 1538 are shown in axial alignment with the shoulder 1544, thus retaining the carriage 1520 in its first position. The fingers 1568 are spaced from the projections 1538. In FIG. 17B, the connection assembly 1500 has been moved further along the axial direction 1501 towards the fingers 1568. Here, the fingers 1568 can contact the projections 1538. As the connection assembly 1500 continues to be moved in the same direction, the contact of the fingers 1568 with the projections 1538 can cause the flexible arms 1537 (shown in FIG. 13 and described above) to deflect away from the axial direction 1501 and cause the projections 1538 affixed to the flexible arms 1537 to move out of axial alignment with the shoulder 1544 on the body 1504. In FIG. 17C, the flexible arms 1537 have been deflected, and the projections 1538 have been removed from axial alignment with the shoulders 1544, by the fingers 1568 on the manifold 1560. Without the engagement between the projections 1538 and the shoulders 1544 to counteract the biasing force applied by the spring 1532, the spring 1532 can now cause movement of the carriage 1520 to the second position within the recess 1540 of the body 1504. When the carriage 1520 is in its second position, the connection assembly 1500 may be in the fully connected configuration, as described above. At this point, the priming process can be initiated to cause priming liquid to be moved between the priming circuit 1300 and the blood circuit 1200. Although FIGS. 17A-17C do not depict a connected priming conduit 1302 to provide better views of the other described components, it will be understood that such a connection should be established prior to starting the priming process.

The cart 1000 may include a plurality of connection assemblies 1500, such that each connector on the blood circuit 1200 can be connected to respective connectors on the priming circuit 1300. In some exemplary embodiments, each of the one or more first end connectors 1216 and second end connectors 1220 of the blood conduit 1202 can be received into, onto, or adjacent to a blood circuit connector 1512 of separate connection assemblies 1500. Similarly, each of the one or more first end connectors 1316 and second end connectors 1320 of the priming conduit 1302 can be received into, onto, or adjacent to a priming circuit connector 1508 of respective separate connection assemblies 1500.

Each connection assembly 1500 may include connectors that are specific to the components being connected to the connection assembly 1500. In some aspects, the first end connector 1316 of the priming conduit 1302 may be different from the second end connector 1320. Accordingly, one connection assembly 1500 that will be utilized with the first end 1308 of the priming conduit 1302 may have a different priming circuit connector 1508 than another connection assembly 1500 that will be utilized with the second end 1312 of the priming conduit 1302. It will be appreciated that the connectors should be complementary to whichever components they are designed to engage with. In an exemplary embodiment depicted in FIGS. 12 and 13, two different priming circuit connectors 1508 are shown, with an exemplary priming circuit connector 1508a referring to a priming circuit connector for use with the first end 1308 of the priming conduit 1302, and an exemplary priming circuit connector 1508b referring to a priming circuit connector for use with the second end 1312 of the priming conduit 1302. It will be appreciated that other suitable connector designs are envisioned. Although FIGS. 12 and 13 depict two priming circuit connectors 1508a and 1508b, it will be understood that each connection assembly 1500 may include a single priming circuit connector, and that the priming circuit connectors may be interchangeable depending on which end of the priming conduit 1302 will be connected to the particular connection assembly 1500. Blood circuit connectors 1512 may also differ based on which portion of the blood conduit 1202 is being connected thereto. In some aspects, a blood circuit connector 1512 for use with a first end connector 1216 may have a smaller cross-sectional dimension than a blood circuit connector 1512 for use with a second end connector 1220. For example, the first and second diameters D1 and D2 shown in FIG. 14C may be smaller in a blood circuit connector 1512 configured to receive a first end connector 1216 of the blood conduit 1202 than respective first and second diameters D1 and D2 of a blood circuit connector 1512 configured to receive a second end connector 1220 of the blood conduit 1202.

In some aspects, it may be advantageous to utilize connection assemblies 1500 that cannot be connected to the wrong conduits. If all connection assemblies 1500 are fully interchangeable with each other, then the risk of user error is increased in that a connector of the blood conduit 1202 may be inadvertently connected to a connector of the priming conduit 1302. To ensure that only the proper connections are made, each connection assembly 1500 may be arranged, or may have components therein that are arranged, in such a manner that only the desired connections can be made, while incorrect connections cannot be successfully made. For example, in some aspects, one or more connection assemblies 1500 that are intended to be used to connect respective first ends 1208 and 1308 of the blood and priming conduits 1202 and 1302 may include blood circuit and priming circuit connectors 1512 and 1508 that only allow proper engagement with the correct first ends 1208 and 1308 and would not permit proper connection with, for example, a second end 1212 of the blood conduit 1202 or a second end 1312 of the priming conduit 1302. Conversely, one or more connection assemblies 1500 that are intended to be used to connect respective second ends 1212 and 1312 of the blood and priming conduits 1202 and 1302 may include blood circuit and priming circuit connectors 1512 and 1508 that only allow proper engagement with the correct second ends 1212 and 1312 and would not permit proper connection with, for example, a first end 1208 of the blood conduit 1202 or a first end 1308 of the priming conduit 1302.

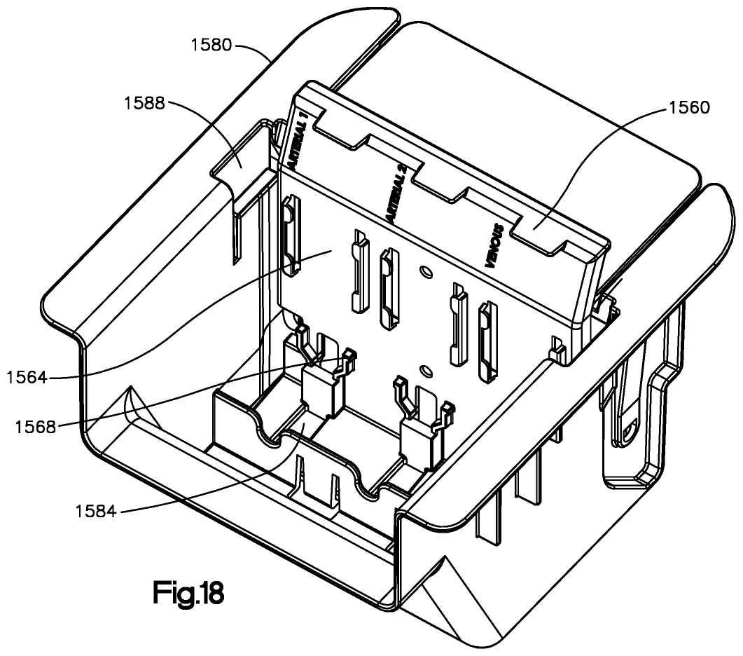

Referring to FIG. 18, the manifold 1560 may be removably retained within a cradle 1580. The cradle 1580 can include an interior receptacle 1584 configured to receive the manifold 1560 (and any attached connection assemblies 1500) therein. The receptacle 1584 may be configured to receive and retain a liquid therein, for example, if any priming liquid leaks out of the priming conduit 1302, the blood conduit 1202, and/or the connection assembly 1500. The cradle 1580 may be releasably connectable to the cart 1000, for example to the upper portion 1004 of the cart 1000 (see, e.g., FIGS. 1-3 and 8).

### Draping Procedure

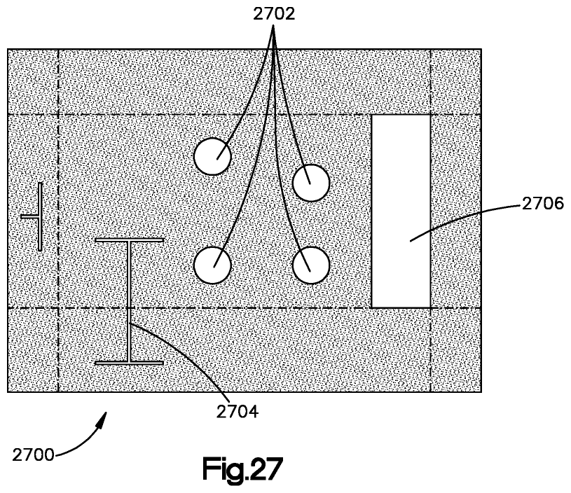

Referring to FIGS. 7 and 27, a drape 2700 can be positioned between the transfer tray 1100 and the cart 1000. The drape 2700 can provide a barrier between sterile and non-sterile components during use of the system. The drape 2700 can provide a thermal barrier between the transfer tray 1100 and the cart 1000. The drape 2702 can include cutouts 2702 for the weight sensors 1008. The drape 2702 can include a cutout 2704 for the connection cradle 1580. The cutout 2704 can be an elongate slit in the drape 2702 such that a flap extends from an upper surface of the cart 1000 into a space between the connection cradle 1580 and the cart 1000. The drape 2700 can include a window 2706 such that the display 1016 is observable through the window 2706 when the drape 2702 is on the cart 1000. The window 2706 can be a transparent film that maintains sterility while allowing light to pass therethrough. The drape 2700 can be detachably coupled to the cart 1000. The connection cradle 1580 can help secure the drape 2700 to the cart 1000.

### Transfer Tray

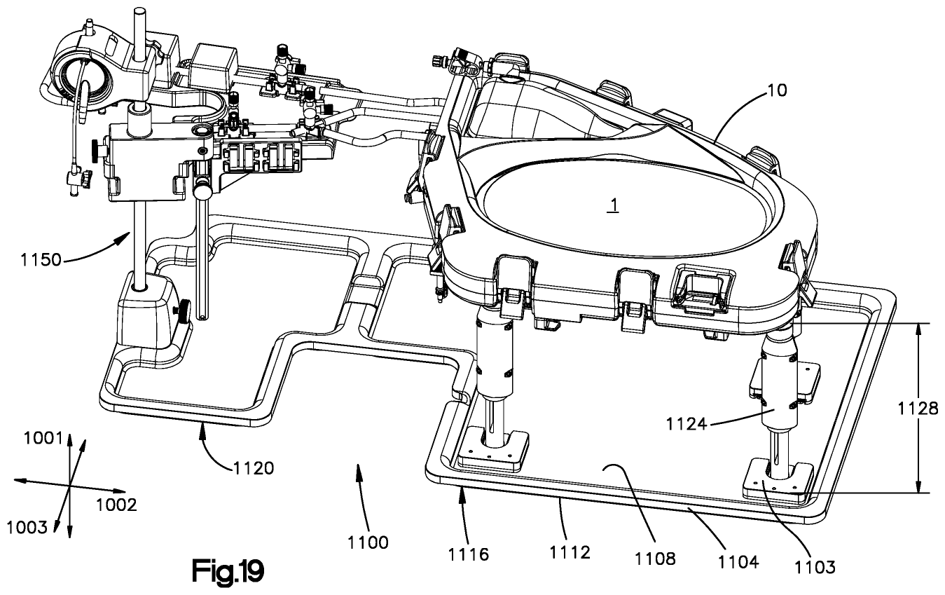

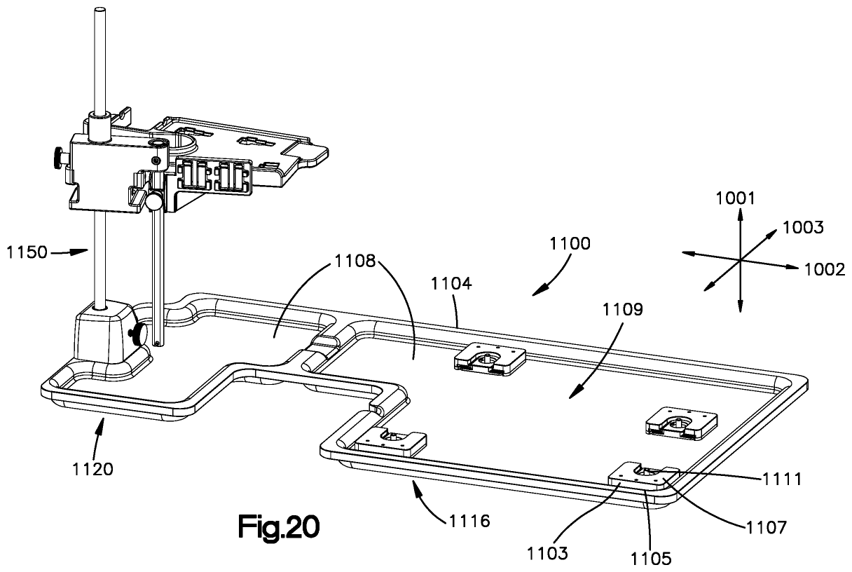

When the blood circuit 1200 is connected with the priming circuit 1300 and is being primed, and when the blood circuit 1200 is being connected to, or is connected to, the neonate, the blood circuit 1200 may be retained on the cart 1000. When the neonate is transferred from the cart 1000 to the main console, the blood circuit 1200 can be transferred along with the neonate (and with the neonatal chamber assembly 10, for example). To facilitate transfer of multiple components along with the neonate, it may be advantageous to utilize a transfer tray 1100 designed to receive and retain the neonate, the neonatal chamber assembly 10, the blood circuit 1200, and/or other components thereon. Referring to FIGS. 19-21, a tray 1100 is depicted for receiving a neonatal chamber assembly 10 with a neonate 1 therein. The tray 1100 includes a body 1104 that defines an upper surface 1108 and a lower surface 1112 opposite the upper surface 1108 and spaced from the upper surface 1108 along the vertical axis 1001. As shown in FIG. 19, the neonatal chamber assembly 10 can be disposed on the body 1104, for example on or adjacent to the upper surface 1108. In some aspects, the upper surface 1108 may be dimensioned such that the tray 1100 is configured to receive a volume of liquid thereon on the upper surface 1108 without allowing the liquid to flow or spill from the tray 1100. As such, the upper surface 1108 may define one or more receptacles, hollows, or grooves 1109 (labeled in FIG. 20) on the body 1104 configured to receive therein the volume of liquid. Such arrangements may allow for capturing spilled liquid in the event of unexpected leakage of the priming liquid, neonatal blood, or neonatal chamber assembly liquid out of the blood circuit 1200 and/or the neonatal chamber assembly 10. In some aspects, the body 1104 of the tray 1100 may be dimensioned such that liquid that overflows from the one or more receptacles 1109 is directed to a desired location relative to the tray 1100, for example, to a designated gutter or waste source (not shown). Such an arrangement may be advantageous to prevent the liquid from contacting any electronic components that may otherwise be damaged by the liquid if the tray 1100 overflows.

The neonate 1 may be introduced into the neonatal chamber assembly 10 either before or after the neonatal chamber assembly 10 has been placed onto the tray 1100. The neonatal chamber assembly 10 can be disposed on a first portion 1116 of the tray 1100. The tray 1100 may include a second portion 1120 that is offset from the first portion 1116 along the longitudinal axis 1002 and/or along the transverse axis 1003. In some aspects, a portion of the first portion 1116 may overlap with a portion of the second portion 1120. For purposes of this disclosure, the first portion 1116 generally refers to the portion of the tray 1100 that is configured to receive the neonate 1 and/or the neonatal chamber assembly 10 thereon, and the second portion 1120 generally refers to the portion of the tray 1100 that is configured to receive a support assembly 1150 for securing the blood circuit 1200 thereto (as will be described in detail below). In some particular aspects, the first portion 1116 may include one or more receptacles 1109, the second portion 1120 may include one or more receptacles 1109, and the one or more receptacles 1109 of the first portion 1116 may be isolated from the one or more receptacles 1109 of the second portion 1120, such that liquid received into the one or more receptacles 1109 of the first portion 1116 is precluded from being mixed with the liquid received into the one or more receptacles 1109 of the second portion 1120.

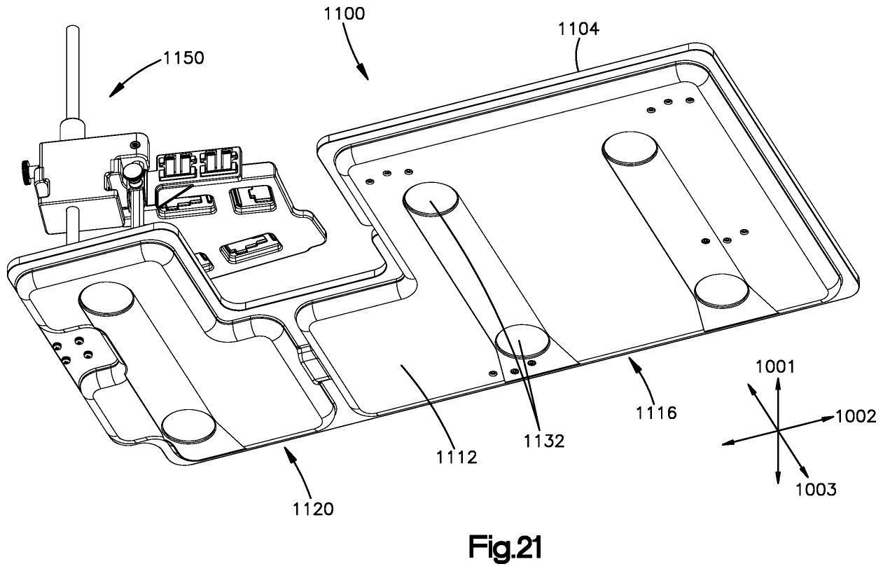

The tray 1100 may be received onto the cart 1000 (see FIG. 1), for example onto the upper surface 1006 of the upper portion 1004. The lower surface 1112 of the tray 1100 may be configured to contact at least a portion of the upper portion 1004 of the cart 1000. In some aspects, as shown in FIG. 21, the tray 1100 may include a retention feature 1132 configured to facilitate desired alignment between the tray 1100 and the cart 1000 and/or to retain the tray 1100 on the cart 1000 to prevent inadvertent movement of the tray 1100 relative to the cart 1000. The retention feature 1132 may include a notch configured to receive a protrusion of a complementary retention feature 1010 on the cart 1000 (labeled in FIG. 2). The retention feature 1010 on the cart 1000 may be adjacent to, or may include, the weight sensor 1008. Additionally, or alternatively, the retention feature 1132 of the tray 1100 may be a protrusion that is receivable within a complementary notch or groove defined on the cart 1000. In some aspects, the retention feature 1132 on the tray 1100 may comprise a material having a higher coefficient of friction than the material of the body of the tray 1100, such that when the tray 1100 is disposed on the cart 1000, the contact between the retention feature 1132 and the cart 1000 along the vertical axis 1001 hinders relative sliding between the tray 1100 and the cart 1000 along the longitudinal and/or the transverse axes 1002 and 1003. In such aspects, the complementary retention feature 1010 on the cart 1000 may comprise such a material having a higher coefficient of friction either in addition to or in place of the retention feature 1132 on the tray 1100. In some aspects, the tray 1100 may include a plurality of retention features 1132 on the lower surface 1112. Accordingly, the cart 1000 may include a plurality of corresponding retention features 1010 thereon. It will be appreciated that the respective arrangement of the retention features 1132 of the tray 1100 and retention features 1010 of the cart 1000 may be arranged so as to allow for a desired orientation and alignment of the tray 1100 relative to the cart 1000.

In some aspects, the neonatal chamber assembly 10 may be disposed directly on the upper surface 1108 of the tray 1100. It should be appreciated that the body 1104 (and particularly the first portion 1116) should be dimensioned and shaped in such a way as to accommodate receiving and securing the neonatal chamber assembly 10. In some aspects, as shown in FIG. 19, for example, the tray 1100 may include a support member 1124 configured to receive the neonatal chamber assembly 10 thereon. The support member 1124 can be affixed to the body 1104 of the tray 1100 via known mechanisms, such as fasteners, clips, welding, friction or wedge fit, or another suitable fixing mechanism. The support member 1124 may be separable and removable form the tray 1100, and the support member 1124 may be replaceable with a different mechanism that is part of the console (not shown). In some aspects, the tray 1100 may be formed having a support member 1124 as an integral portion of the unitary tray 1100.

In some aspects, the support member 1124 may have a height 1128 measured along the vertical axis 1001 from the upper surface 1108 along the support member 1124. The height 1128 of the support member 1124 may be adjustable to allow the support members 1124 to be shortened and removed when the neonatal chamber assembly 10 has been secured to a support mechanism that is part of the console (not shown). Alternatively, the height 1128 of the support member 1124 may be adjustable based on the desired distance between the body 1104 of the tray 1100 and the neonatal chamber assembly 10 or the neonate 1. In some aspects, the support member 1124 may include a releasable retention mechanism (not shown) configured to releasably affix to the neonatal chamber assembly 10 to prevent inadvertent separation of the neonatal chamber assembly 10 from the support member 1124. In some aspects, the support member 1124 may be shaped or dimensioned such that a portion of the support member 1124 is received into a complementary notch or groove (not shown) on the neonatal chamber assembly 10, or, additionally or alternatively, the support member 1124 may define such a notch or groove configured to receive a complementary protrusion defined on the neonatal chamber assembly 10.

In some aspects, the tray 1100 may be configured to receive a plurality of support members 1124, such as 2, 3, 4, 5, 6, 7, or more support members 1124. In some particular embodiments disclosed herein, the tray 1100 may include four support members 1124. Each of the support members 1124 may be disposed on the body 1104 such that together, all support members 1124 can receive the neonatal chamber assembly 10 thereon and retain the neonatal chamber assembly 10 in a stable and secure manner relative to the rest of the tray 1100. The support members 1124 can be detachably coupled to the tray 1100. The tray 1100 can include a receiver 1103 adapted to receive a portion of the support member 1124. The receiver 1103 can include a sidewall 1105 and an upper wall 1107 that define a recess to receive a portion of the support member 1124. The receiver 1103 can include a pin 1111 adapted to be received in a recess of the support member 1124. The pin 1111 can be a spring-loaded pin that is movable with respect to the receiver 1103 such that a foot of the support member 1124 can slide horizontally into the recess defined by the receiver 1103. The neonatal chamber assembly 10 can be coupled to a support assembly 1150 such that the neonatal chamber assembly 10 can be supported without using the support member 1124. The support members 1124 can be individually detachable from the neonatal chamber assembly 10. The neonatal chamber assembly 10 can be cantilevered from the support assembly 1150.

In some aspects, the tray 1100 may include a preferred color, pattern, or other visual scheme to provide a contrast backdrop to facilitate identifying, isolating, and visualizing the neonate in the neonatal chamber assembly 10 when the tray 100 is disposed in the main console.

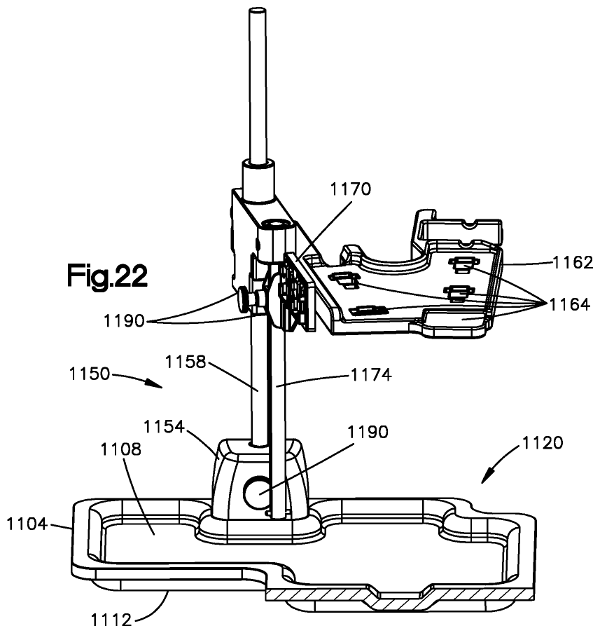

The tray 1100 can include a gantry or support assembly 1150 for receiving, retaining, and/or manipulating one or more components of the blood circuit 1200. The support assembly 1150 may be disposed in the second portion 1120 of the tray 1100. With continued reference to FIGS. 19-21 and with further reference to FIG. 22, a support assembly 1150 may include a base 1154, via which the support assembly 1150 can be attached to the tray 1100. The base 1154 may be part of an integral uniform tray 1100, or the base 1154 can alternatively be a separable component. The base 1154 may be attached to the tray 1100 via a known connection mechanism, such as fasteners, clips, welding, friction or wedge fit, or another suitable fixing mechanism. The base 1154 may receive a post 1158 therein or thereon. The post 1158 may be secured to the tray 1100 via the base 1154 and may extend from the base 1154, at least in part, along the vertical axis 1001 away from the tray 1100. The post 1158 can be movable with respect to the base 1154 and the tray 1100. The post 1158 may be movable along the vertical axis 1101 toward or away from the base 1154. In some aspects, the post 1158 may be rotatable around the vertical axis 1001 (or around another axis parallel to the vertical axis 1001). The support assembly 1150 may be configured to selectively permit or preclude translation and/or rotation of the post 1158 relative to the base 1154. A retention device 1190 may be disposed on the base 1154 and/or on the post 1158 that can be actuated to selectively permit and preclude movement of the post 1158. In some aspects, the retention device 1190 may be a knob 1190 that can be rotated in a first direction to secure the post 1158 with respect to the base 1154 to prevent movement of the post 1158, and that can be rotated in a second direction opposite the first direction to allow movement of the post 1158. This disclosure is not limited to the particular retention device utilized, and other envisioned embodiments of the support assembly 1150 may include other suitable retention mechanisms, such as clips, pins, ties, or other fasteners. In some aspects, the post 1158 may be rigidly fixed relative to the base 1154 such that the post 1158 is not movable relative to the base 1154. In some aspects, the post 1158 may integrated with the base 1154 as part of a unitary support assembly 1150.

A movable support body 1162 is disposed on the post 1158. The support body 1162 may be configured to receive at least a portion of the blood circuit 1200 thereon. The support body 1162 may include thereon, or may receive thereon, one or more attachment members 1164 configured to releasably affix a portion of the blood circuit 1200 to the support body 1162. The one or more attachment members 1164 may include clips, tethers, tube conduits or channels, tie-downs, grooves or notches, or other suitable components configured to retain portions of the blood circuit 1200 on the support body 1162 without causing damage to the blood circuit 1200 and without interfering with the desired operation of the blood circuit 1200 or its related components. The support body 1162 may be movable relative to the tray 1100 along the post 1158. The support body 1162 may be translatable along the vertical axis 1001 towards and away from the tray 1100. The support body 1162 may be rotatable around the post 1158 (i.e., around the vertical axis 1001 or around an axis parallel to the vertical axis 1001). In some aspects, the support body 1162 may be rotated up to 360 degrees around the post 1158. The translation and/or rotation of the support body 1162 allows for adjustment of the blood circuit 1200 relative to the neonatal chamber assembly 10 and/or the priming circuit 1300. The maneuverability of the support body 1162 can also allow for unobstructed, or less obstructed, access to components on the tray 1100 that would otherwise be difficult to access if the support body 1162 were not permitted to move. For example, the support body 1162 may be in a first position while the neonate is being cannulated to provide additional space for the user to cannulate the neonate, and then in a second position translationally and/or rotationally offset from the first position after the neonate has been cannulated and the flow of liquid through the blood circuit 1200 and the neonate has been established. Other positions of the support body 1162 are also envisioned, for example during priming of the blood circuit 1200 (as described above) or during replacement and/or priming of the oxygenator 800 (as will be described further below). Another retention device 1190 may be disposed on the post 1158, on the support body 1162, or on both, to selectively permit translational and/or rotational movement of the support body 1162 relative to the post 1158. The retention device 1190 may any suitable retention mechanism as described above.

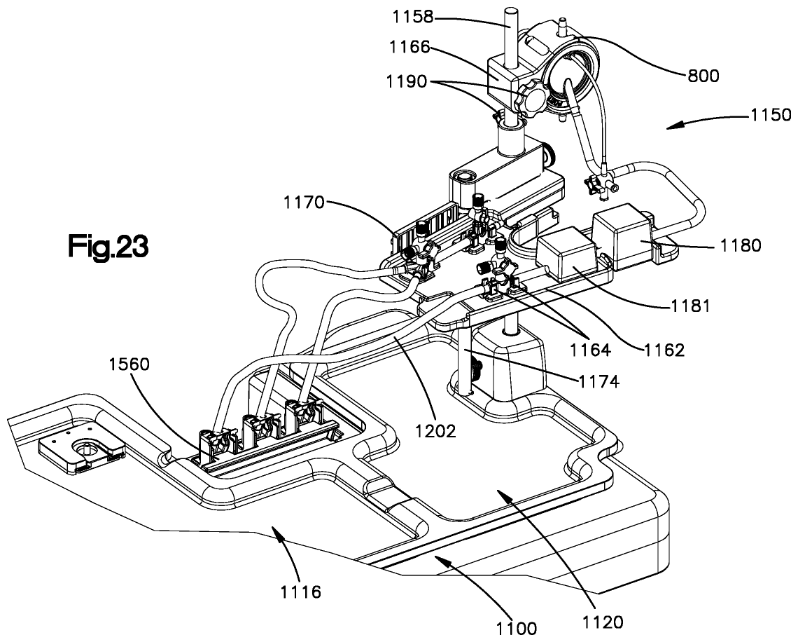

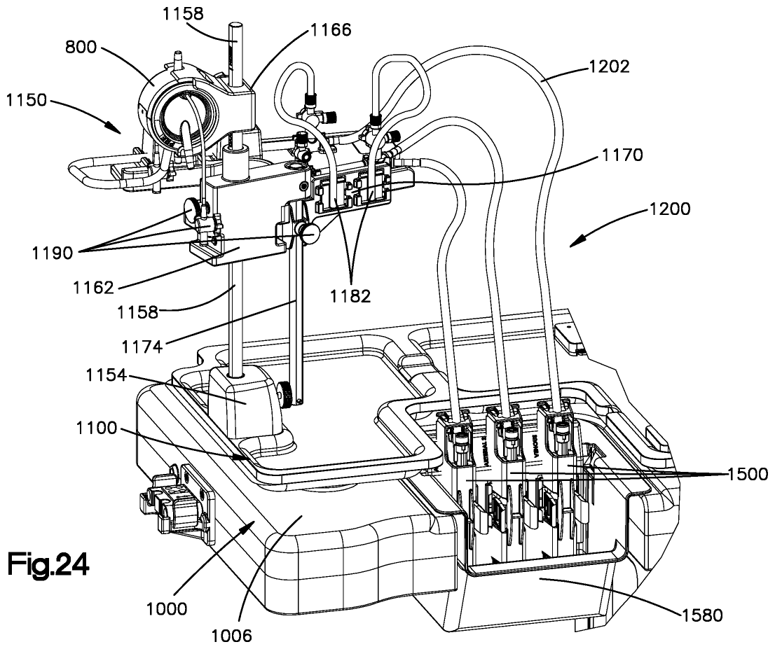

The support assembly 1150 may be configured to receive and/or retain thereon one or more sensors configured to monitor, measure, record, and/or transmit information related to various parameters of the blood circuit 1200, the priming liquid, the neonatal blood, the neonate, environment in the neonatal chamber assembly 10, and/or the ambient environment around the tray 1100 and/or the cart 1000. Blood may be moved through the blood circuit 1200 by the neonate's heart and, therefore, the one or more sensors can monitor performance of the neonate's heart. Sensors can include temperature sensors, flow meters, pressure sensors, gas saturation sensors, light sensors, air bubble sensors, liquid composition sensors, meconium sensors, and other sensors for detecting desired parameters related to neonatal development. One or more of the sensors can be configured to communicate with a controller and processor (not shown) that may receive and record measurements, perform calculations related to the received measurements, and/or transmit signals or instructions based on the received data to other components of the tray 1100, the cart 1000, the blood circuit 1200, the priming circuit 1300, or another system connected physically or wirelessly. With continued reference to FIG. 22 and with further reference to FIGS. 23 and 24, the one or more sensors may be retained on the support body 1162. As shown in an exemplary embodiment of FIG. 23 and in an alternative exemplary embodiment of FIG. 24, a blood gas saturation sensor or flow sensor 1180 may be disposed on the support body 1162 and be configured to measure one or more gas saturation of the liquid flowing through the blood conduit 1202. The gas saturation sensor 1180 may be configured to measure a saturation of oxygen gas in the liquid flowing through the blood conduit 1202. In some embodiments, a plurality of gas sensors 1180 may be disposed to measure gas saturation along various portions of the blood circuit 1200, for example, a first gas saturation sensor disposed on the first blood conduit portion 1204 for measuring oxygen saturation in liquid flowing towards the oxygenator 800 pre-oxygenation and a second gas saturation sensor 1180 disposed on the second blood conduit portion 1206 for measuring oxygen saturation in liquid flowing away from the oxygenator 800 post-oxygenation. The one or more gas saturation sensors 1180 may be non-invasive, such that the one or more gas saturation sensors 1180 can operate without being inserted into, or otherwise penetrating, the blood conduit 1202. For example, the one or more gas saturation sensors 1180 may be designed to be disposed adjacent to the blood conduit 1202.

One or more flow sensors 1181 may be disposed on the support body 1162 and configured to detect parameters of liquid and/or gas flow through the blood conduit 1202. The one or more flow sensors 1181 may be configured to detect the rate of liquid flow, as well as other characteristics, such as viscosity, turbulence, and composition. In some aspects, the one or more flow sensors 1181 may be configured to detect and/or quantify presence of air bubbles within the liquid flowing through the blood conduit 1202. Detection of air bubbles may be preferred to allow for identification and removal of the air bubbles to decrease risk of injury to the neonate.

One or more pressure transducers or sensors 1182 may be disposed on or adjacent to the support body 1162 that are configured to measure the liquid pressure within the blood conduit 1202. In some aspects, the one or more pressure sensors 1182 may be configured to measure pressure within a blood vessel of the neonate. In some particular embodiments, the one or more pressure sensors 1182 may be configured to measure the arterial pressure. In the exemplary aspects of FIGS. 23 and 24, two separate pressure sensors 1182 are depicted. One of the two pressure sensors 1182 may be configured to detect pressure measurements within the first blood conduit portion 1204, and the other of the two pressure sensors 1182 may be configured to detect pressure measurements within the second blood conduit portion 1206.

In some aspects, it may be preferred to be able to adjust the position of the pressure sensors 1182 relative to the tray 1100 based on the respective position of the neonate 1. The pressure sensors 1182 may be configured to be moved along the vertical axis 1001 toward or away from the tray 1100 based on the position of the neonate 1. The height of the pressure sensors 1182 (measured along the vertical axis 1001 between the upper surface 1108 of the tray 1100 and the pressure sensors 1182) may be adjusted to correspond the neonate's phlebostatic axis. This may be advantageous to allow for approximating the neonate's mean arterial pressure. In some aspects, the neonate 1 may be moved relative to the pressure sensors 1182, for example, by adjusting the height 1128 of the neonatal chamber assembly 10 by adjusting the height of the support members 1124. In preferred aspects, the pressure sensors 1182 may be moved relative to the neonate 1. The pressure sensors 1182 may be moved by moving one or more components of the support assembly 1150. In some aspects, the support body 1162 may be moved along the post 1158 toward or away from the tray 1100, such that the pressure sensors 1182 are placed in substantially the same plane, defined by the longitudinal and transverse axes 1002 and 1003, as the neonate 1, and specifically, the neonate's heart.

In some more preferred embodiments, the pressure sensors 1182 may be movable along the vertical axis 1001 relative to the support body 1162 without requiring movement of the support body 1162 itself. Referring again to FIG. 22, a pressure sensor holder 1170 may be operably connected to the support assembly 1150, for example, to the support body 1162. The pressure sensor holder 1170 is configured to receive the one or more pressure sensors 1182, such as the two pressure sensors 1182 in some disclosed embodiments. The plurality of pressure sensors 1182 may all be arranged in the same plane defined by the longitudinal and transverse axes 1002 and 1003. The pressure sensor holder 1170 may be configured to be slidably movable along the vertical axis 1001 relative to the tray 1100. In some aspects, the pressure sensor holder 1170 may be slidably movable relative to the support body 1162. As shown in FIG. 22, for example, a rail 1174 may be disposed on the support body 1162, along which the pressure sensor holder 1170 may be moved. It will be appreciated that other suitable arrangements may be envisioned that permit the plurality of pressure sensors 1182 to be moved along the vertical axis 1001. One or more retention devices 1190 may be disposed on the support assembly 1150, on the support body 1162, on the pressure sensor holder 1170, and/or on the rail 1174 configured to selectively allow and preclude translational movement of the pressure sensor holder 1170 (and the pressure sensors 1182 thereon). The one or more retention devices 1190 may be one or more of the different types of suitable retention devices 1190 described above.

In some aspects, the support assembly 1150 may be configured to receive and retain the oxygenator 800 thereon. Referring to FIGS. 22-24, an oxygen retainer 1166 may be disposed on the support assembly 1150. The oxygen retainer 1166 is configured to releasably secure an oxygenator 800. In some aspects, the oxygen retainer 1166 may be disposed on the support body 1162 and may be movable relative to the tray 1100 when the support body 1162 is moved. In other aspects, the oxygen retainer 1166 may be affixed to the post 1158 and may be separate from the support body 1162. The oxygenator retainer 1166 may be translatable along the post 1158 along the vertical axis 1001 toward and away from the tray 1100. The oxygenator retainer 1166 may be rotatable around the post 1158. In some aspects, a retention device 1190 may be disposed on the support assembly 1150 and configured to selectively allow or preclude translation and/or rotation of the oxygen retainer 1166. The retention device 1190 may be any one of the suitable retention devices described above. In some aspects, one of the previously disclosed retention devices 1190 configured to allow or preclude translation and/or rotation of one or more of the other disclosed components of the support assembly 1150 (e.g., the post 1158, the support body 1162, and/or the pressure sensor holder 1170) may additionally be configured to allow or precluded translation and/or rotation of the oxygenator retainer 1166. During use, the oxygenator 800 may be moved relative to the rest of the blood circuit 1200 and relative to other components of the tray 1100 and/or the support assembly 1150. The oxygenator 800 may be moved by rotating or translating the oxygen retainer 1166 with the oxygenator 800 therein. For example, during cannulation of the neonate, the oxygenator 800 may be moved to a first position, such that the user performing the cannulation has unobstructed access to the neonate 1 and other components of the blood circuit 1200 on the tray 1100. After cannulation has been performed, the oxygenator 800 may be moved to a second position that is translationally and/or rotationally offset from the first position, such that the oxygenator 800 is disposed in the desired orientation and arrangement relative to the blood circuit 1200. In some aspects, when the oxygenator 800 is being primed and prepared for use (or when a replacement oxygenator is being primed and prepared for use), the oxygenator 800 may similarly be moved to the first position and then moved to the second position upon completion of the priming process (as will be described below).

In some aspects, the support assembly 1150 may be configured to retain the components described above such that formation of bubbles within those components is minimized. For example, the connectors related to the blood circuit 1200 described above (e.g., the one or more access ports 1224) may be arranged on the support assembly 1150 in a substantially vertical orientation (i.e., substantially perpendicular to the vertical axis 1001) to facilitate removal of trapped air within those components. Trapped air, being less dense than the liquid within the blood circuit 1200, will be displaced by the liquid in the blood circuit 1200, as the liquid is acted on by gravity, and thus the trapped air will be above the liquid in each component (relative to ground and along the vertical axis 1001). By aligning the connectors vertically, the trapped air may be more accessible and more easily removed via the one or more connectors.

### Oxygenator Swap and Priming

In some aspects, the oxygenator 800 that is configured for use with the blood circuit 1200, as described above, needs to be replaced with a different oxygenator. This may be due to damage or clogging of the oxygenator 800 over time. In some cases, after the blood circuit 1200 has been connected to the neonate and the neonate's blood is being circulated through the blood circuit 1200, the oxygenator 800 may need to be replaced with a replacement oxygenator 800a. In such cases, it may be preferable to maintain as much of the circulation functionality of the blood circuit 1200 as possible while the oxygenator replacement is performed. If the blood circuit 1200 is disconnected from the neonate, or if blood flow through the blood circuit 1200 is stopped, this could cause adverse effects to the neonate. As such, it may be advantageous to perform the oxygenator replacement as quickly as possible and without stopping flow of the blood through the blood circuit 1200 and the neonate.

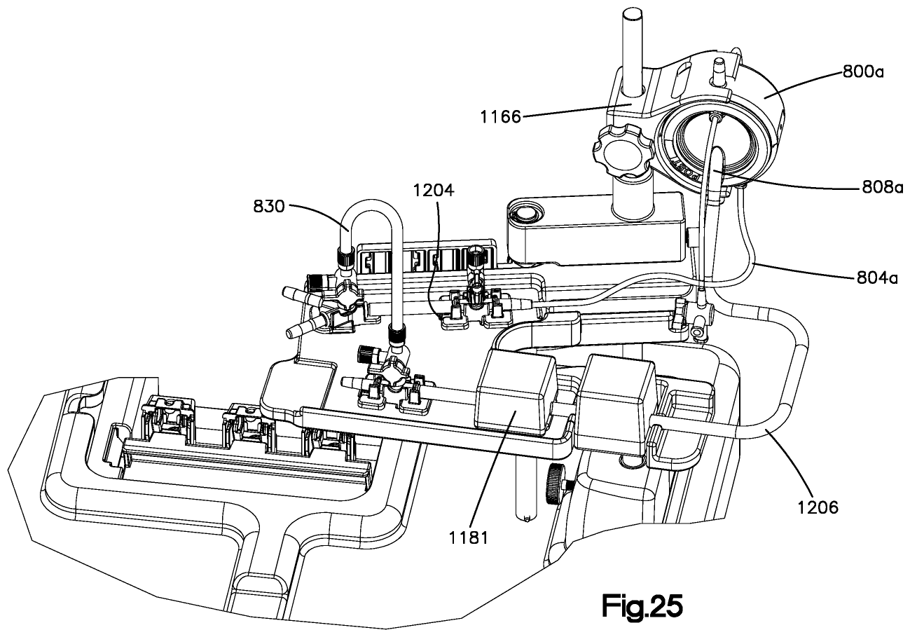

To introduce another oxygenator into the blood circuit 1200, the new oxygenator needs to be prepared, primed, and conditioned. In some aspects, similar components that can be used to prime the blood circuit 1200 as described above can also be used to prime a new oxygenator. The new oxygenator can be primed before the initial oxygenator 800 is removed from the blood circuit 1200. Referring to FIG. 25, a replacement oxygenator 800a may be connected to the priming circuit 1300 on the cart 1000. The priming circuit 1300 in such embodiments can be substantially similar to, or the same as, embodiments of the priming circuit 1300 described elsewhere in this application, and components of the cart 1000 and oxygenator 800 may be substantially the same as those described earlier unless noted otherwise.

FIG. 25 shows a replacement oxygenator 800a having a liquid inlet 804a, a liquid outlet 808a, a gas inlet 812a, and a gas outlet 820a. The replacement oxygenator 800a and its components may be substantially similar to the embodiments of the oxygenator 800 and its respective components described throughout this disclosure. A gas conduit 820 is configured to connect the gas inlet 812a to the gas source, such as a gas tank 1040 (labeled in FIGS. 2 and 3) or another suitable gas source, such as a valve, faucet, or spout fixture (not shown). The replacement oxygenator 800a may be received and retained relative to the cart 1000 on a replacement oxygenator retainer 1166a. The replacement oxygenator retainer 1166a may be releasably connectable to the cart 1000, for example, to the upper portion 1004.

FIG. 27 depicts a flow chart illustrating an exemplary method 1800 of priming a replacement oxygenator 800a and replacing the oxygenator 800 with the primed replacement oxygenator 800a. In step 1804, the replacement oxygenator 800a can be disposed on the cart 1000 and connected to the priming circuit 1300. It will be appreciated that the priming process can be performed elsewhere, but due to the integrated components on the cart 1000 related to the priming circuit 1300 for use with priming the blood circuit 1200 and the original oxygenator 800, it may be advantageous to utilize the same systems already in place to also prime the replacement oxygenator 800a. The replacement oxygenator 800a may be secured to the cart 1000 in any suitable manner, such as via the replacement oxygenator retainer 1166a. One of the first end 1308 and the second end 1312 of the priming conduit 1302 may be connected to the liquid inlet 804a of the replacement oxygenator 800a. The other of the first end 1308 and the second end 1312 of the priming conduit 1302 may be connected to the liquid outlet 808a. The priming conduit 1302 may be substantially the same as the priming conduit 1302 described above. The connections between the priming conduit 1302 and the liquid inlet 804a and liquid outlet 808a may include a barbed tube connector. It will be appreciated that other suitable connections are envisioned.

In step 1808, the priming liquid is introduced into the priming conduit 1302. As explained previously, the priming liquid can be moved from the priming liquid source 1331 via the priming liquid supply line 1330. Excess priming liquid can be moved out of the priming conduit 1302 via the waste line 1334 into the waste receptacle 1335.

In step 1812, the priming liquid in the priming conduit 1302 can be circulated through the priming conduit 1302 towards the replacement oxygenator 800a. The priming liquid can be moved into the replacement oxygenator 800a at the liquid inlet 804a, through the replacement oxygenator 800a, and then out through the liquid outlet 808a. The priming liquid can then be returned to the priming conduit 1302. Movement of the priming liquid can be caused by actuation of the pump 1052, and the priming liquid may be heated by the heater 1048, as described elsewhere in this application.

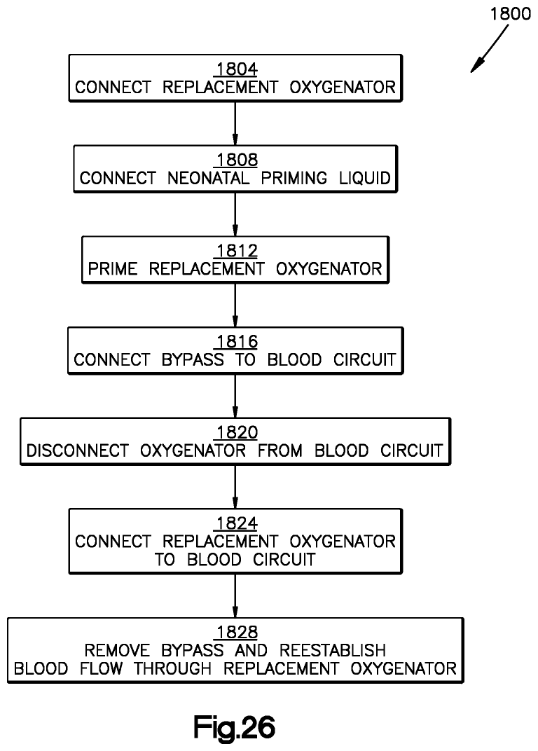

After the replacement oxygenator 800a has been sufficiently primed, the original oxygenator 800 may be removed and replaced from the blood circuit 1200 that is connected to the neonate. Prior to disconnecting the oxygenator 800 from the blood circuit 1200, it may be preferable to maintain the blood flow through the blood circuit 1200, even if the blood is not passing through the oxygenator 800. Referring to FIG. 26, in step 1816, a bypass 830 can be introduced and connected to the blood circuit 1200 to temporarily divert the blood flowing through the blood conduit 1202 around the oxygenator 800 in step 1820. The bypass 830 may be a tube or conduit similar in composition and/or material to the blood conduit 1202. At one end thereof, the bypass 830 is configured to be connected to the blood conduit 1202 upstream of the oxygenator 800 (i.e., at the first portion 1204 of the blood conduit 1202, between the first end 1208 and the liquid inlet 804). At the other end thereof, the bypass 830 is configured to be connected to the blood conduit 1202 downstream of the oxygenator 800 (i.e., at the second portion 1206 of the blood conduit 1202, between the liquid outlet 808 and the second end 1212 of the blood conduit 1202). When connected, the bypass 830 should be in liquid communication with the blood conduit 1202, such that the neonatal blood flowing from the first end 1208 towards the oxygenator 800 is received from the first portion 1204 of the blood conduit 1202 into the bypass 830, moved through the bypass 830, and is discharged from the bypass 830 into the second portion 1206 of the blood conduit 1202. In such a manner, blood flow can continue from the neonate through the blood circuit 1200 and back to the neonate while the oxygenator 800 is being replaced. The bypass 830 should be dimensioned and comprise materials that substantially match flow resistance to that of the oxygenator so that parameters of the liquid flowing through the blood circuit 1200 when the bypass 830 is connected are similar to those of the liquid flowing through the blood circuit 1200 when the oxygenator 800 is connected.

With continued reference to FIG. 26, in step 1820, the oxygenator 800 connected to the blood circuit 1200 can be taken out of communication with the blood conduit 1202. This can be accomplished by clamping a portion of the blood conduit 1202 at the first portion 1204 (for example, adjacent to the liquid inlet 804) and also clamping a portion of the blood conduit 1202 at the second portion 1206 (for example, adjacent the liquid outlet 808). It will be understood that other mechanisms can be utilized to prevent blood flow through the oxygenator 800 in the blood circuit 1200. Step 1820 can be performed after the bypass 830 has been connected to the blood conduit 1202. The oxygenator 800 can then be separated from the blood conduit 1202, for example by severing portions of the blood conduit 1202 adjacent the liquid inlet 804 and the liquid outlet 808. Similarly, the gas conduit 820 connected to the gas inlet 812 may be disconnected from the oxygenator 800. Any other conduits can be disconnected from the oxygenator 800 as well (e.g., an additional gas conduit 820 connected to the gas outlet 816). The oxygenator 800 may be removed from the oxygenator retainer 1166 on the support assembly 1150.

In step 1824, the primed replacement oxygenator 800a can be introduced into the oxygenator retainer 1166 or elsewhere on the support assembly 1150. The blood conduit 1202 may be connected to the replacement oxygenator 800a. For example, the first portion 1204 of the blood conduit 1202 can be connected to the liquid inlet 804a, and the second portion 1206 of the blood conduit 1202 can be connected to the liquid outlet 808a. The connection may be made via any known mechanism, for example via barbed connectors between conduits or tubes. The one or more gas conduits 820 can be connected to the gas inlet 812a and/or the gas outlet 816a.

In step 1828, after the replacement oxygenator 800a has been connected to the blood circuit 1200 in place of the initial oxygenator 800, the blood flow can be introduced through the replacement oxygenator 800a. Any clamps or other blocking devices that were introduced in step 1820 can be removed such that the neonatal blood can flow from the neonate into the blood conduit 1202 at the first end 1208, through the first portion 1204 into, through, and out of the replacement oxygenator 800a, through the second portion 1206, and out of the blood conduit 1202 at the second end 1212 back into the neonate. The bypass 830 can be taken out of liquid communication with the blood conduit 1202 and removed from the blood circuit 1200.

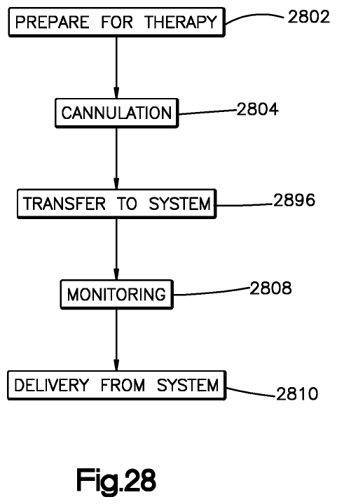

Referring to FIG. 28, a method of operating the system is shown. Step 2802 includes preparing for therapy. Step 2802 can include providing power to the cart 1000, the neonate chamber assembly 10, and the blood circuit 1200. The oxygenator 800 can be connected to supply gas (e.g., wall gas or gas tank 1040). An ultrasound can be performed while the neonate is in the womb to measure the umbilical vessel diameter to determine appropriate cannula sizing. The priming circuit 1300 can prime the blood circuit 1200 during step 2802. The priming circuit 1300 can prime the blood circuit 1200 while the transfer tray 1100 is on the cart 1000. The transfer tray 1100 can be positioned on the cart 1000 before the neonate is introduced into the neonate chamber assembly 10.

Step 2804 can include performing a cesarean section on the mother with an ultrasound guided paralytic administered to the neonate prior to delivery to prevent the neonate from taking a first breath. While Step 2804 is described in the context of a cesarean section, a vaginal delivery can also be used with the method and system described herein. The umbilical cord can be cut, and the neonate can be transferred to a partially filled and warmed saline solution in the neonate chamber assembly 10. The neonate's umbilical cord can be cannulated to connect the neonate to a blood circuit 1200 to allow blood to flow from the neonate into and through the blood circuit 1200 and back into the neonate. The lid of the neonate chamber assembly 10 can then be closed and locked to the base. Additional saline solution can then be added to the neonate chamber assembly 10.

Step 2806 can include moving the transfer tray 1100 onto the cart 1000. Any sensors (pressure, temperature, meconium detector, blood detector) coupled to the neonate chamber assembly 10 can begin to transmit signals to another device or computer. An ultrasound can be performed when the neonate is within the neonate chamber assembly 10. The cart 1000 can then be transferred to the neonate intensive care unit.

Step 2808 can include checking vital signs, fetal growth assessments, and camera images of the neonate. Blood samples can be taken from the blood circuit 1200 during step 2808. The blood samples can be drawn without opening the neonatal chamber assembly 10. The system can be periodically calibrated during step 2808 via gas analyzers and pressure sensors.

At step 2810, a clinical decision can be made to deliver the neonate from the neonate chamber assembly 10 into the standard of care. The lid of the neonate chamber assembly 10 can be unlocked and removed from the base. Excess saline solution can be removed from the neonate chamber assembly 10. The umbilical cord can be clamped and cut. The neonate can then be delivered to standard of care therapy.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

When values are expressed as approximations by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, reference to values stated in ranges includes each and every value within that range.

Throughout this specification, words are to be afforded their normal meaning as would be understood by those skilled in the relevant art. However, so as to avoid misunderstanding, the meanings of certain terms will be specifically defined or clarified.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

1. A transfer tray for receiving a neonate thereon, the transfer tray comprising:
a body having a first portion configured to receive a neonatal chamber assembly thereon, the neonatal chamber assembly configured to receive the neonate therein, and a second portion configured to receive a blood circuit thereon, the blood circuit comprising an oxygenator, a first conduit for transfer of blood from the neonate to the oxygenator, and a second conduit for transfer of blood from the oxygenator to the neonate, the first conduit having an arterial end portion configured to be placed in liquid communication with an artery of an umbilical cord of the neonate, and the second conduit having a venous end portion configured to be placed in liquid communication with a vein of the umbilical cord; and
a movable support assembly affixed to the body of the tray and configured to receive the blood circuit thereon,
wherein, when the neonate is disposed on the first portion and is in liquid communication with the blood circuit, the transfer tray is movable from a first position, in which the transfer tray is disposed on a first surface, to a second position, in which the transfer tray is disposed on a second surface different from the first surface.

2. The transfer tray of claim 1, wherein the first surface includes a priming cart, and the second surface includes an incubator.

3. The transfer tray of claim 1, further comprising a support member configured to receive the neonatal chamber assembly thereon, the support member causing the neonatal chamber assembly to be spaced from the transfer tray at a first height.

4. The transfer tray of claim 3, wherein the support member is adjustable between having a first height and a second height different from the first height, the first and second heights being measured between the transfer tray and the neonatal chamber assembly.

5. The transfer tray of claim 3, comprising a plurality of support members.

6. The transfer tray of claim 1, wherein the body defines a receptacle defined thereon configured to receive and retain a first volume of liquid.

7. The transfer tray of claim 1, wherein the support assembly comprises a support body attached thereto, the support body configured to receive at least one sensor out of the group of oxygen sensor, flow meter, temperature sensor, pressure sensor, and bubble sensor.

8. The transfer tray of claim 7, wherein the support body is movable relative to the support assembly.

9. The transfer tray of claim 1, wherein the support assembly comprises an oxygenator retainer configured to releasably receive the oxygenator thereon.

10. The transfer tray of claim 9, wherein the oxygenator retainer is movable relative to the support assembly.

11. The transfer tray of claim 1, further comprising a first and a second pressure sensor disposed on the support assembly, the first pressure sensor being configured to measure pressure within the first conduit and the second pressure sensor being configured to measure pressure within the second conduit.

12. The transfer tray of claim 11, wherein the first and second pressure sensors are movable along a vertical axis relative to the transfer tray toward and away from the transfer tray.

13. The transfer tray of claim 11, wherein the neonate is configured to be disposed in a first plane spaced from the transfer tray along the vertical axis, and the first and second pressure sensors are configured to be moved into the first plane.

14. The transfer tray of claim 1, further comprising a retention member on the body of the transfer tray, the retention member configured to perform at least one of:
aligning the transfer tray with at least one of the first surface and the second surface; and
precluding slidable movement of the transfer tray relative to at least one of the first surface and the second surface.

15-38. (canceled)

39. A method of cannulating an umbilical cord of a neonate on a priming apparatus, the priming apparatus comprising a blood circuit comprising an arterial end, a venous end opposite the arterial end, a blood conduit extending between the arterial and venous ends, and an oxygenator disposed in line with the conduit between the arterial end and the venous end, the priming apparatus further comprising a priming circuit comprising a first end, a second end opposite the first end, and a priming conduit extending between the first and second ends, wherein the first end of the priming conduit is configured to releasably connect with the arterial end of the blood conduit, and the second end of the priming conduit is configured to releasably connect with the venous end of the blood conduit, such that the blood conduit is in liquid communication with the priming conduit, the blood circuit and the priming circuit being configured to receive a priming liquid, the method comprising:
providing the neonate on an upper surface of the priming apparatus;
calculating a weight of the neonate via a weight sensor on the upper surface of the priming apparatus;
connecting the arterial end of the blood conduit to an artery in the umbilical cord of the neonate; and
connecting the venous end of the blood conduit to a vein in the umbilical cord of the neonate,
wherein, when at last one of the arterial and the venous ends of the blood conduit are connected to the umbilical cord, the blood circuit is not in liquid communication with the priming circuit.

40. A method of priming a blood circuit with a priming liquid, the blood circuit being configured to be connected to a neonate, the blood circuit comprising a first end, a second end opposite the first end, a blood conduit extending between the first and second ends, and an oxygenator disposed in line with the blood conduit, the method comprising:
connecting the first end of the blood circuit to a first end of a priming conduit of a priming circuit, such that the blood conduit and the priming conduit are in liquid communication with each other;
connecting the second end of the blood conduit to a second end of the priming conduit, the second end being spaced from the first end of the priming conduit, the priming conduit extending between its first and second ends;
receiving the priming liquid into the priming conduit from a priming liquid source;
actuating movement of the priming liquid within the priming conduit into the blood conduit by pumping the priming liquid toward the connected blood conduit;
after receiving the priming liquid into the blood conduit, discharging the priming liquid from the blood conduit into the priming conduit, the priming liquid having passed through the blood conduit and the oxygenator prior to being discharged into the priming conduit; and
heating the priming liquid by contacting a heater to the priming conduit.

41. The method of claim 40, wherein the priming liquid is pumped through the priming conduit towards one of the first end and the second end into the respective connected first end and second end of the blood conduit, and the priming liquid is discharged into the priming conduit from the blood conduit at the other of the first end and the second end of the priming conduit.

42. The method of claim 40, wherein the pumping of the priming liquid is actuated by a peristaltic pump configured to operably contact the priming conduit.

43. The method of claim 40, further comprising moving the priming liquid from the priming liquid source into the priming conduit through a supply line connecting the priming liquid source to the priming conduit.

44. The method of claim 40, further comprising introducing a gas into the oxygenator at a gas inlet, wherein at least a portion of the gas is introduced into the priming liquid as the priming liquid is moved through the oxygenator.

45. The method of claim 40, further comprising measuring a temperature of the priming liquid in at last one of the priming conduit and the blood conduit.

46. The method of claim 45, further comprising providing a signal to the heater to increase or decrease heat based on the measured temperature.

47. The method of claim 40, wherein the priming liquid includes at least one of blood plasma from the neonate, blood plasma from a human that is not the neonate, and synthetic plasma.

48. The method of, wherein the priming liquid is a first priming liquid, and the method further comprises introducing a second priming liquid into the priming conduit from a second priming liquid source.

49. The method of claim 48, wherein the first priming liquid includes a crystalloid solution, and the second priming liquid includes human blood.

50-56. (canceled)
